(19) United States
(12) Patent Application Publication
(10) Pub. No.: US 2015/0141822 A1
(43) Pub. Date: May 21, 2015

(54) METHOD FOR SETTING REGIONS OF INTEREST AND ULTRASOUND DIAGNOSTIC APPARATUS

(71) Applicant: HITACHI ALOKA MEDICAL, LTD., Mitaka-shi, Tokyo (JP)

(72) Inventors: Yusuke Miyauchi, Mitaka-shi (JP); Hirotaka Baba, Mitaka-shi (JP); Takashi Iimura, Mitaka-shi (JP); Naoyuki Murayama, Mitaka-shi (JP); Koji Waki, Mitaka-shi (JP)

(21) Appl. No.: 14/405,686

(22) PCT Filed: May 20, 2013
(86) PCT No.: PCT/JP2013/063941

§ 371 Date: Dec. 4, 2014

(30) Foreign Application Priority Data
Jun. 7, 2012 (JP) 2012-130139
Feb. 12, 2013 (JP) 2013-024812
Mar. 29, 2013 (JP) 2013-074714

(51) Int. Cl. A61B 8/00; A61B 8/08; A61B 8/14; G01N 29/44

(52) U.S. Cl. CPC A61B 8/469; G01N 29/44; A61B 8/5223; A61B 8/5207; A61B 8/14; A61B 8/54; A61B 8/461; A61B 8/485; A61B 8/481
USPC 600/438

(57) ABSTRACT

A method for generating a region of interest (ROI) wherein in setting ROIs in biological tissues to be compared, burden on a subject can be reduced and the reproducibility of elasticity measurement improved, including: setting candidate points in an arbitrary designated region in a notable tissue in a contrast image of an object designated by an input device; determining partial differential values of pixel values in a two-dimensional direction in the contrast image and thus detecting a tissue boundary; acquiring a shortest distance between the detected tissue boundary and each point, and setting a circle or polygonal region inscribed in the circle having the maximum shortest distance as a radius around the candidate point having the maximum shortest distance as a region of interest; and imaging the region of interest and superimposing it on the contrast image and displaying it on an image display unit.

### TECHNICAL FIELD

The present invention relates to an ultrasound diagnostic apparatus provided with a function for displaying an elasticity image indicating hardness or softness of a biological tissue of an object. Particularly, the present invention relates to a method for setting regions of interest and an ultrasound diagnostic apparatus suitable for measurement of an elasticity value (strain or an elastic modulus, for example) of a plurality of regions of interest (hereinafter referred to as an ROI) set for a region for comparing hardness or softness of a biological tissue, respectively, and evaluation of elasticity by a ratio of the elasticity value (hereinafter referred to as an elastic ratio) of those regions to be compared.

### BACKGROUND ART

As a method for designating two points which are a point of a substantial center of a closed region surrounded by a tissue boundary and a point on the tissue boundary by the ultrasound diagnostic apparatus and for automatically tracing the tissue boundary so as to set an ROI on an image, the one described in Patent Literature 1, for example, can be cited. Moreover, as a method for displaying a cross-section region image and an elasticity image indicating hardness or softness of a biological tissue in order to promote improvement of diagnostic accuracy, for calculating the respective elasticity values of a region of interest set in a tumor portion (tumor ROI) and a region of interest set in a fat portion (fat ROI), and for displaying a ratio of these elasticity values (elastic ratio) so as to make contribution to diagnosis of benignity or malignancy of a tumor, necessity of a surgery and the like, the one described in Patent Literature 2, for example, can be cited.

### CITATION LIST

### Patent Literature

Patent Literature 1: JP 4607263

Patent Literature 2: JP 3991282

### SUMMARY OF INVENTION

### Technical Problem

If setting of an ROI can be made semi-automatically as in Patent Literatures 1 and 2, the number of labors of an examiner can be decreased, examination time can be reduced, intervention by manual work can be made less, and reproducibility of measured values can be improved, and that is a useful function. However, it has problems as described below.

That is, according to Patent Literature 1, it is necessary to designate at least two spots for setting one ROI, and that is still cumbersome. Moreover, if the tissue boundary image is missing or is not closed, constitution of a closed region becomes difficult, and setting of an appropriate ROI becomes difficult. Moreover, if the size of an ROI is not more than a certain level, sampling regions run short and measured values might become errors and in such a case, setting of an ROI should be made again. Moreover, in Patent Literature 2, since ROI setting is made manually, reproducibility of elasticity values in the tumor ROI and the fat ROI is low, and there is a concern that accuracy of a final elastic ratio is also lowered. If setting of the ROI should be made again for that, labor and time required for the ROI setting becomes a burden both for an examiner and an object.

A problem to be solved by the present invention is to provide a method for setting an ROI which can reduce a burden for an examiner when an ROI is to be set for biological tissues to be compared and has high reproducibility of a measured value and an ultrasound diagnostic apparatus using the method for setting.

### Solution to Problem

In order to solve the above-described problems, a method for generating an ROI of the present invention comprises a first step for setting a plurality of candidate points in an arbitrary designated region designated in a notable tissue in an ultrasound image of an object by an input device, a second step for calculating a change of a pixel value in a two-dimensional direction of the ultrasound image and for detecting a tissue boundary, a third step for acquiring a shortest distance between the detected tissue boundary and each of the candidate points and for setting a circle or a regular polygonal region inscribed in the circle having the shortest distance which is a longest thereof as a radius around the candidate point having the shortest distance which is the longest thereof as a region of interest, and a fourth step for imaging the region of interest which was set and superimposing it on the ultrasound image and for displaying it on an image display portion. As a result, a region of interest (ROI) with a wide area can be automatically generated.

Moreover, in order to solve the above-described problems, an ultrasound diagnostic apparatus of the present invention includes a probe for transmitting an ultrasonic wave to an object and for receiving a reflected signal from the object, a transmission and reception unit configured to transmit and receive ultrasonic waves to or from the object by driving the probe and for executing signal processing of the reflected signal, an image generation unit configured to generate an ultrasound image by using the reflected signal subjected to the signal processing, a display configured to display the ultrasound image, and a control panel on which an arbitrary parameter is set by an operator for generating the ultrasound image, in which a first reference position included in a first diagnostic region of the displayed ultrasound image is set by the control panel, and the image generation unit is provided with a region generation portion for generating a second diagnosis region to be generated on the ultrasound image by using positional information of the first diagnosis region, protrusion to an outside of the ultrasound image, and edges and peripheral tissues of the first diagnosis region. As a result, when one of regions of interest in a comparative relationship is generated, the other region of interest can be automatically generated.

In order to solve the above-described problems, the present invention is a method for setting a region of interest, in order to calculate a ratio of elasticity values (strain or elastic modulus) between a first region of an ultrasound image obtained by the ultrasound diagnostic apparatus and a second region with a biological tissue different from that of the first region, for setting a first region of interest in the first region and for setting a second region of interest in the second region, in which a reference region of interest having an area determined in advance is generated at a position designated as the first region on the ultrasound image, the reference region of interest is enlarged and the first region of interest is generated and set, the second region of interest is generated and set for the second region, the elasticity values (strain or elastic modulus, for example) of the first region of interest and the second region of interest which are set, respectively, are calculated, respectively, it is evaluated whether generation of the first region of interest and the second region of interest is appropriate or not on the basis of each of the elasticity values or a ratio thereof, and at least one of the first region of interest and the second region of interest is modified (a position or an area is modified, for example) in accordance with the evaluation. As a result, a burden for the examiner when an ROI is set in a biological tissue to be compared can be reduced, and an ROI with high reproducibility of elasticity measurement can be set.

### Advantageous Effects of Invention

According to the present invention, a burden for the examiner when an ROI is set in a biological tissue to be compared can be reduced, and an ROI with high reproducibility of a measured value can be set.

### BRIEF DESCRIPTION OF DRAWINGS

FIG. 1 is a configuration diagram of an ultrasound diagnostic apparatus of Embodiment 1 of the present invention.

FIG. 2 is a configuration diagram of an ROI generation unit 12 in FIG. 1.

FIG. 3 is a flowchart illustrating an example of a processing procedure of an ROI setting unit of a feature portion of Embodiment 1.

FIG. 4 are views for explaining a specific example of an ROI set by the ROI setting unit of Embodiment 1.

FIG. 5 are views for explaining an example of an operation for setting the ROI using a method for setting an ROI in Embodiment 1.

FIG. 6 are views for explaining another example of the operation for setting the ROI using the method for setting an ROI in Embodiment 1.

FIG. 7 are views for explaining still another example of the operation for setting the ROI using the method for setting an ROI in Embodiment 1.

FIG. 8 are views for explaining an example of the operation for setting the ROI when a designated region is set to an elliptic region in the method for setting an ROI in Embodiment 1.

FIG. 9 are views for explaining another example of the method for setting an ROI when the designated region having a two-dimensional shape is input/set.

FIG. 10 is a block configuration diagram of an ultrasound diagnostic apparatus according to Embodiment 2 of the present invention.

FIG. 11 is a block diagram exemplifying a configuration of a region generation unit of Embodiment 2.

FIG. 12 are views schematically illustrating a setting procedure of a tumor ROI in a first ROI generation unit of Embodiment 2.

FIG. 13 are views schematically illustrating conditions for generating possibility distribution of Embodiment 2, the generated possibility distribution, and a fat ROI which is a second diagnosis region generated by using the possibility distribution.

FIG. 14 is a flowchart illustrating an outline of a processing procedure in the ultrasound diagnostic apparatus according to Embodiment 2.

FIG. 15 is a flowchart illustrating an example of a procedure for generating the fat ROI in the region generation unit of Embodiment 2.

FIG. 16 is a block configuration diagram of an ultrasound diagnostic apparatus of Embodiment 3 of the present invention.

FIG. 17 is a flowchart illustrating a processing procedure of a region of interest generation unit of Embodiment 3.

FIG. 18 is a view for explaining a display example of a display screen of Embodiment 3.

FIG. 19 are views for explaining an operation 1 of the region of interest generation unit of Embodiment 3.

FIG. 20 are views for explaining an operation 2 of the region of interest generation unit of Embodiment 3.

FIG. 21 are views for explaining an operation 3 of the region of interest generation unit of Embodiment 3.

FIG. 22 are views for explaining an operation 4 of the region of interest generation unit of Embodiment 3.

FIG. 23 are views for explaining a variation of an ROI shape of Embodiment 3.

FIG. 24 are views for explaining a processing example of the region of interest generation unit of Embodiment 3.

FIG. 25 are views for explaining another processing example of the region of interest generation unit of Embodiment 3.

### DESCRIPTION OF EMBODIMENTS

Embodiments of the present invention will be explained below in detail by referring to the attached drawings.

### Embodiment 1

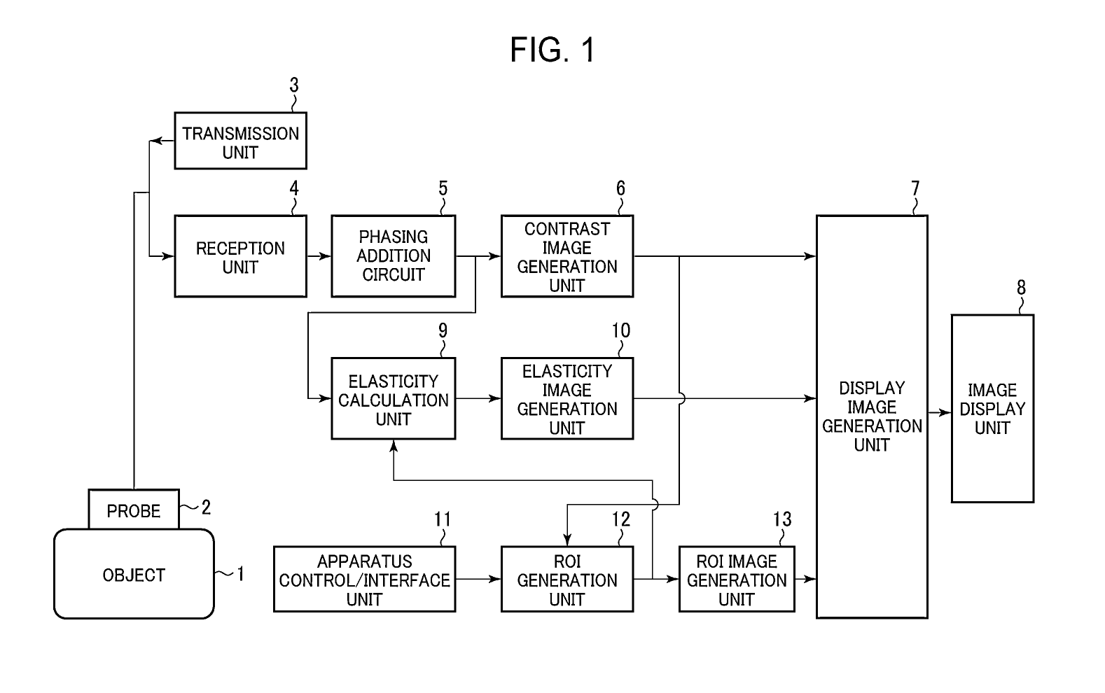

An ultrasound diagnostic apparatus of Embodiment 1 is characterized by automatic generation of a region of interest which is as wide as possible in a notable tissue for which a nature of a biological tissue is to be measured and is constituted as illustrated in FIG. 1. In FIG. 1, a probe 2 converts an ultrasound signal given by a transmission unit 3 to an acoustic signal and sends it to an inside of an object 1. The acoustic signal reflected from the inside of the object 1 (hereinafter referred to as a reflected echo signal) is converted to an electric signal and is transmitted to a reception unit 4. The reception unit 4 applies reception processing to the reflected echo signal having been converted to the electric signal and outputs it to a phasing addition circuit 5. The phasing addition circuit 5 forms a reception beam signal of the reflected echo signal and outputs it to a contrast image generation unit 6. The contrast image generation unit 6 is configured to generate a contrast image called a B-mode image in general, on the basis of the reception beam signal and to display it on an image display unit 8 through a display image generation unit 7.

Moreover, in this embodiment, in addition to the B-mode contrast image, an elasticity image of the biological tissue is generated and displayed on the image display unit 8 through the display image generation unit 7. That is, for example, while a pressure force applied by the probe 2 to the object 1 is being changed, the B-mode contrast image is taken. Regarding the pressure applied to the object, known pressure methods can be applied in addition to pulsation and beating. An elasticity calculation unit 9 inputs a reception beam signal of the B-mode contrast image outputted from the phasing addition circuit 5 and stores frame data of the B-mode contrast image in a time series. Then, a pair of frame data with different photographing time is read out from the stored frame data, and an elasticity value of the tissue is acquired on the basis of displacement of the tissue caused by a difference in pressures. As the elasticity value, an elastic modulus can be typically acquired on the basis of the strain in addition to the strain (percentage). The elasticity calculation unit 9 outputs the frame data of the elasticity value acquired for each of measurement points (pixels) to an elasticity image generation unit 10. The elasticity image generation unit 10 is configured to generate an elasticity image made into a color image on the basis of the elasticity value frame data and to display it on the image display unit 8 through the display image generation unit 7.

On the other hand, an apparatus control/interface unit 11 is capable of controlling the transmission unit 3, the reception unit 4, the phasing addition circuit 5, the contrast image generation unit 6, the display image generation unit 7, the elasticity calculation unit 9, and the elasticity image generation unit 10 and of making various settings, though not shown for simplification of illustration. Particularly, it instructs an input and control of an instruction required for an ROI generation unit 12 which is a feature portion of the present invention. The ROI generation unit 12 sets a region of interest (ROI) on the basis of the inputted instruction and outputs coordinate data of the ROI to the elasticity calculation unit 9 and an ROI image generation unit 13. The elasticity calculation unit 9 acquires only the elasticity value in the ROI generated by the ROI generation unit 12, and the elasticity image generation unit 10 is configured to generate only the elasticity image in the ROI and to be capable of displaying it on the image display unit 8 through the display image generation unit 7. The ROI image generation unit 13 is configured to image a designated point or a designated region which is inputted from the apparatus control/interface unit 11, which will be described later, and to generate the ROI image on the basis of the coordinate data of the ROI outputted from the ROI generation unit 12.

The display image generation unit 7 can display the contrast image outputted from the contrast image generation unit 6 and the elasticity image outputted from the elasticity image generation unit 10 on the image display unit 8 individually in accordance with a control instruction of the apparatus control/interface unit 11. Moreover, it can superimpose and display those images on the image display unit 8. Furthermore, it is configured to superimpose and display the designated point or the designated region imaged by the ROI image generation unit 13 and the ROI image on the contrast image and/or the elasticity image of the image display unit 8 through the display image generation unit 7.

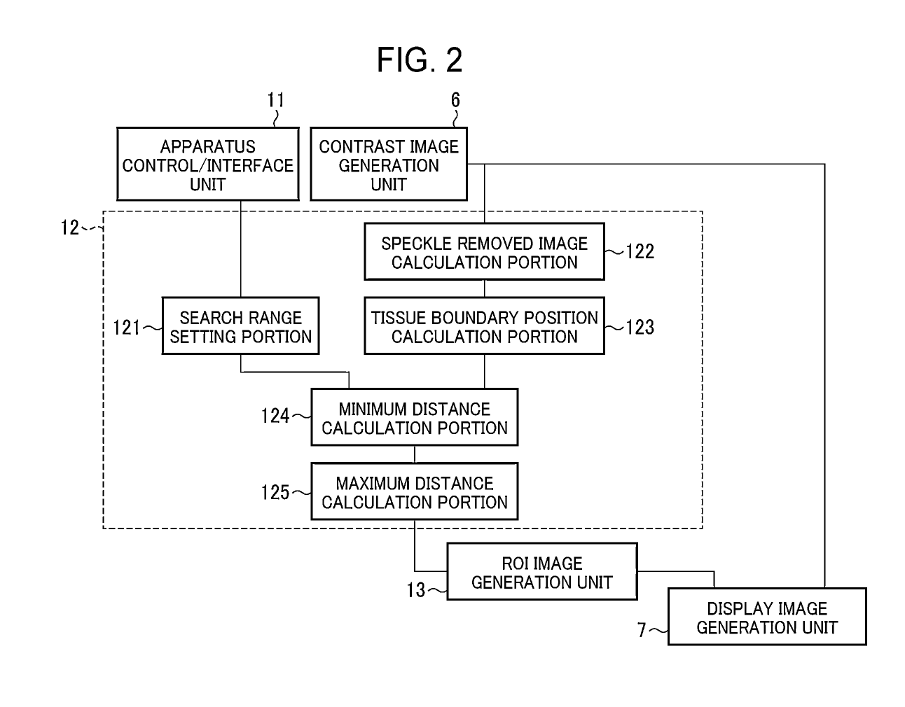

FIG. 2 illustrates a detailed configuration of the ROI generation unit 12. The designated position on the tissue image designated by the examiner by using the apparatus control/interface unit 11 is given to a search range setting portion 121. The search range setting portion 121 calculates a plurality of points within a range of a radius r₀ determined in advance around the designated position (hereinafter referred to as center candidate points) and gives it to a minimum distance calculation portion 124. The contrast image generation unit 6 gives a contrast image to a speckle removed image calculation portion 122. The speckle removed image calculation portion 122 removes a so-called speckle which is an interference fringe in an ultrasound image from the contrast image and gives it to a tissue boundary position calculation portion 123. The tissue boundary position calculation portion 123 calculates a boundary (profile) position of a tissue from an image form which the speckle is removed and gives it to the minimum distance calculation portion 124. The minimum distance calculation portion 124 gives a minimum distance of each of the center candidate points in the distances between the candidate points and the tissue boundary to a maximum distance calculation portion 125. The maximum distance calculation portion 125 selects a point indicating a maximum distance from the center candidate points and gives it to the ROI image generation unit 13. The ROI image generation unit 13 generates an image of a region of interest and gives it to the display image generation unit 7.

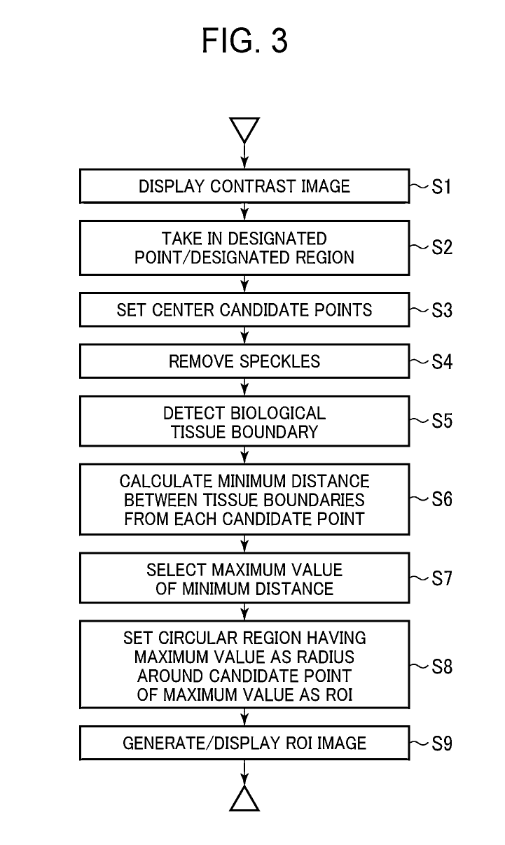

A processing operation relating to ROI setting of the ROI generation unit 12 of the embodiment configured as above is illustrated in a flowchart of FIG. 3. First, the apparatus control/interface unit 11 has the image display unit 8 display a contrast image through the display image generation unit 7 by sending an instruction to the contrast image generation unit 6 (S1). Then, the ROI generation unit 12 takes in coordinate data of a designated point or a designated region inputted by the examiner in the contrast image in the image display unit 8 from the apparatus control/interface unit 11 into the search range setting portion 121 by using an input device such as a position designating device which is an input unit provided in the apparatus control/interface unit 11 (S2). The designated region can be a circular region having a radius r₀ determined in advance around an arbitrary designated point designated by the input device or a two-dimensional region designated by drawing by the input device. Here, it is only necessary that the two-dimensional region is an arbitrary closed figure and may be a rectangle, an ellipse or a regular polygon, for example. When, when coordinates of the designated point are inputted, a plurality of candidate points are set in the whole region of the circular designated region with the radius r₀ determined in advance (S3). Candidate points are set at positions corresponding to pixels of the contrast image. On the other hand, if the coordinate data of the two-dimensional region is inputted as a designated region, a plurality of candidate points are set for the whole area of a designated region 25 (S3). If there is a speckle in the contrast image, the apparatus control/interface unit 11 sends an instruction to the contrast image generation unit 6, so that the speckle removed image calculation portion 122 executes speckle removing processing of the contrast image (S4) and outputs the contrast image from which the speckle is removed to the tissue boundary position calculation portion 123. Here, the speckle removal is processing for removing an interference fringe, that is, a so-called speckle in an ultrasound image from the contrast image, and a known LEE filter or a bilateral filter, for example, can be used.

Subsequently, the ROI generation unit 12 detects a boundary of a biological tissue in which a designated point P0 is set by using the contrast image from which the speckle is removed (S5). As the boundary detecting method of a biological tissue, a known technology can be used. For example, as a first method, a pixel value such as brightness of a pixel in the contrast image is acquired along a search line set radially from each of the candidate points, and a change of the pixel value is acquired along the search line by partial differentiation. On the basis of a distribution image of the acquired partial differential value, a pixel with the partial differential value at a threshold value determined in advance or more is acquired, and the tissue boundary line is detected. That is, a so-called ridge of gradient corresponds to the tissue boundary where, if the properties of adjacent biological tissues are the same, the partial differential value of the pixel value in the search direction is small, while if the properties of the adjacent biological tissues are different, an absolute value of the partial differential value of the pixel value in the search direction becomes large on its boundary. As a second method, by convolving a Sobel operator, for example, in a brightness value of the pixel of the contrast image from which the speckle is removed, by acquiring partial differential values in a lateral direction and a vertical direction on an image plane and by acquiring a square-root of sum of squares of the partial differential value in each direction, distribution of a gradient length of the brightness can be obtained. The gradient length of the brightness can be calculated by using an absolute value of the partial differential value of the brightness. It is expressed that a spot with a large brightness difference has a long gradient length, while a spot with zero brightness difference has no gradient length. Thus, the gradient of the brightness of the contrast image is calculated, the gradient length and a gradient direction are acquired from the gradient, and a spot which becomes a ridge of the gradient length when seen in the gradient direction can be detected as a tissue boundary.

Subsequently, the minimum distance calculation portion 124 calculates a distance dij (j is a natural number from 1 to m.) from each of candidate points P1 (i is a natural number from 1 to n.) to the tissue boundary and calculates minimum distances dimin between them, respectively (S6). The maximum distance calculation portion 125 selects a maximum value dkmax from all the calculated minimum distances dimin (S7). A circular region having the maximum value dmax as a radius around a candidate point Pk of the maximum value dmax is set as a region of interest (ROI), and ROI coordinate data is outputted to the ROI image generation unit 13 (S8). The ROI image generation unit 13 generates an ROI image and outputs it to the display image generation unit 7, and the display image generation unit 7 superimposes the ROI on the contrast image and displays it on the image display unit 8 (S9).

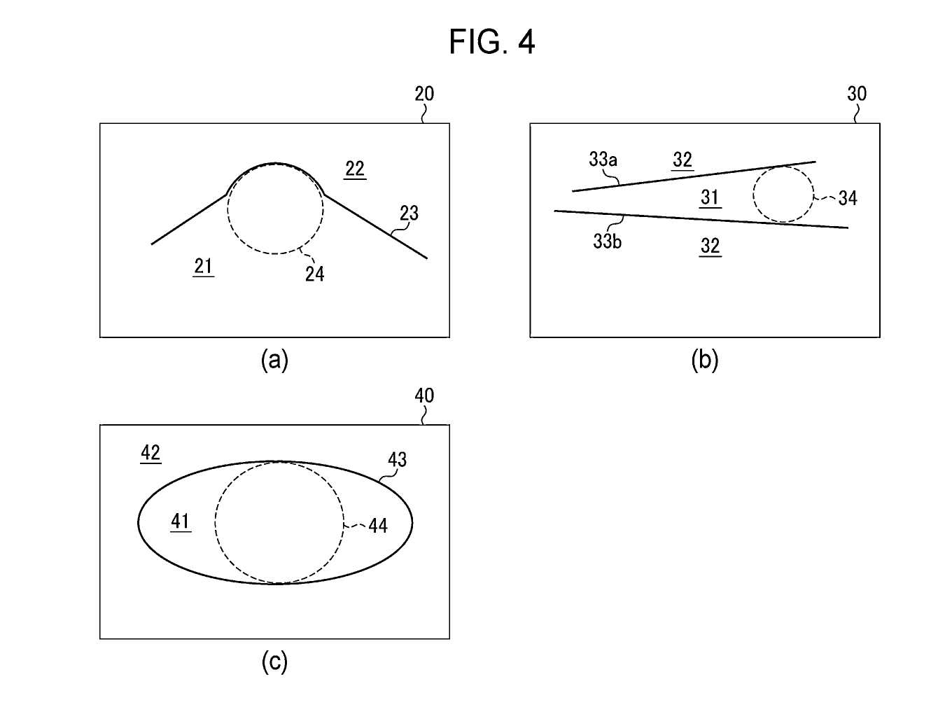

Here, a generation operation of an ROI will be explained by using an example of a specific contrast image. FIG. 4 illustrate examples of a contrast image as an ROI setting target. FIG. 4(a) is an example of a figure in which a boundary line 23 of another biological tissue 22 adjacent to a biological tissue 21 as a notable tissue in a contrast image 20 is not closed. In the case of this example, a tissue property of a portion on a lower side in the figure of the biological tissue 21 is preferably uniform, but the examiner pays attention to a convex part at the center part of the biological tissue 21 of the contrast image 20. In this case, a circular ROI 24 is set having a radius as large as possible conforming to the convex part. FIG. 4(b) is an example in which a fat layer 31 is set as a notable tissue in a contrast image 30 and illustrates a state in which the fat layer 31 is layered and is adjacent with another biological tissue (fat layer and the like) 32 through boundary lines 33a and 33b. In this case, a circular ROI 34 is set as a circle having a radius as large as possible in a region sandwiched by the boundary lines 33a and 33b. FIG. 4(c) illustrates a state in which a biological tissue 41 as a notable tissue in a contrast image 40 is adjacent to another biological tissue 42 through an elliptic boundary line 43. In this case, a circular ROI 44 is set as a circle having a radius as large as possible in a region surrounded by the boundary line 43.

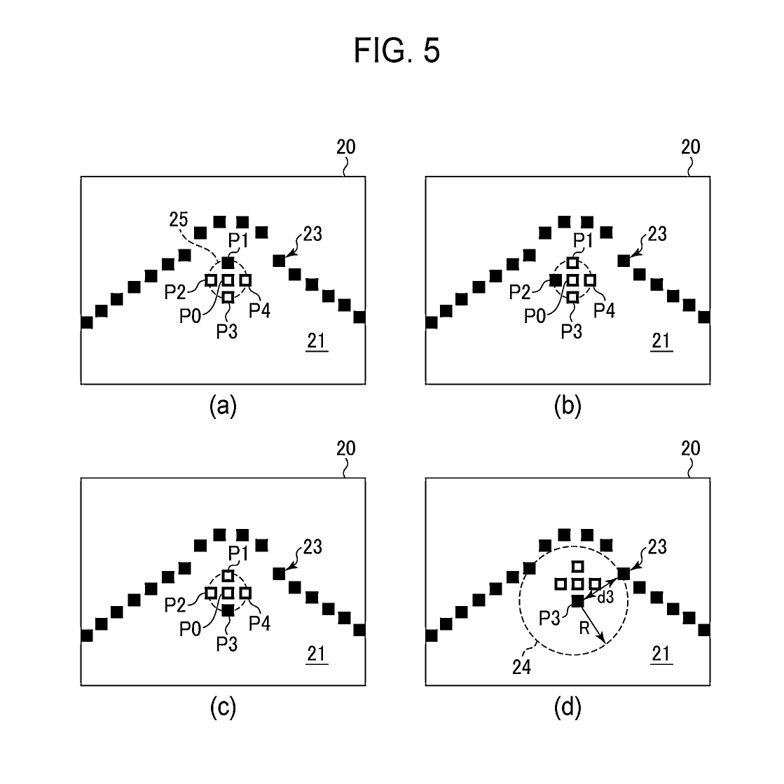

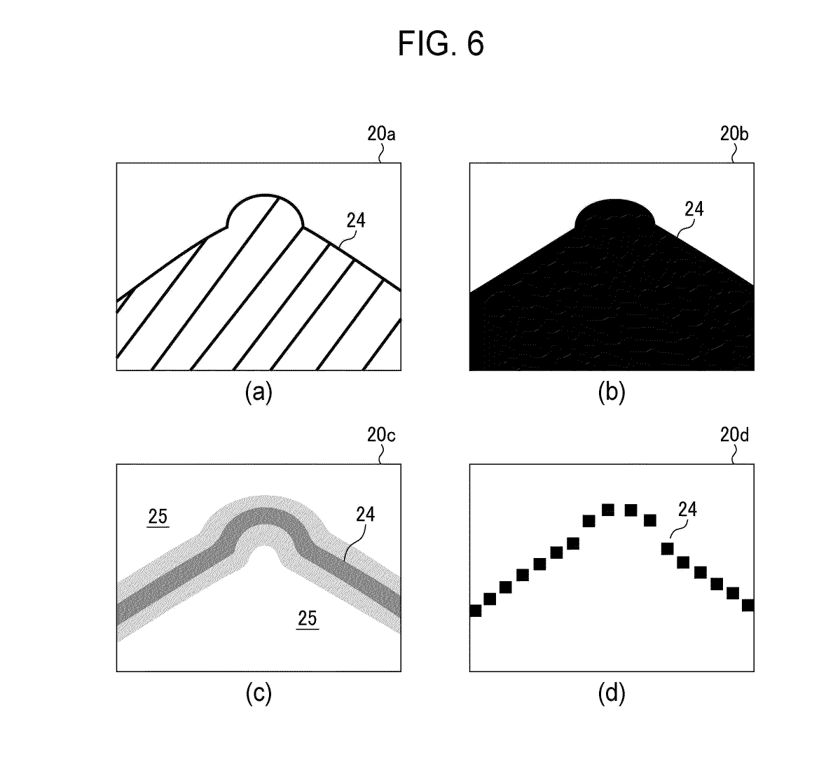

First, by referring to the example of the contrast image 20 in FIG. 4(a), the ROI setting operation will be specifically explained. In FIG. 5, the boundary line 23 between the biological tissue 21 which is a notable tissue of the contrast image 20 detected in the tissue boundary detection step S5 and the adjacent biological tissue 22 is expressed by black square pixels. The boundary line 23 is detected by the above-described second boundary detection method. That is, as illustrated in FIG. 6(a), the speckles are removed from a contrast image 20a given by the contrast image generation unit 6 and a contrast image 20b illustrated in FIG. 6(b) is obtained. In the contrast image 20a, the biological tissue 21 as the notable tissue is schematically illustrated with diagonal lines given, and the contrast image 20b schematically expresses that brightness in a white region is higher than in a blackened region and the brightness in each region is uniform. FIG. 6(c) illustrates a distribution image 20c of gradient lengths detected in the tissue boundary detection step (S5). This figure illustrates that the gradient length in a black spot 25 is longer than that in a white spot 24, and the gradient length at a spot with a brightness difference is long, and the gradient length of a region without brightness difference is zero. FIG. 6(d) is ridge pixel distribution 20d expressing a pixel at the ridge position of the gradient length by a black square. Moreover, the black squares are points on pixels and are juxtaposed adjacent to each other. Moreover, coordinates of each pixel are determined in advance. The ridge of the gradient length is a convex spot when seen in a gradient direction as is known, and by comparing a gradient length pixel in the gradient direction and a pixel value of the gradient length pixel in an anti-gradient direction at a pixel position of each gradient length, and if the gradient length of interest has the longest value, it is made a ridge, so that the tissue boundary 23 can be acquired as a ridge.

Subsequently, a processing operation of ROI setting will be specifically explained by referring to FIG. 5. First, as illustrated in FIG. 5(a), the examiner sets a designated point P0 in an interest region 24 to be diagnosed in the contrast image 20 displayed on the image display unit 8 by using a pointing device of the apparatus control/interface unit 11. It is only necessary that this designated point P0 is set substantially at a center part of the interest region 24. Subsequently, the ROI generation unit 12 sets a designated region of a circle with a radius r₀ set in advance around the designated point P0 and sets a plurality of candidate points P1 inside the designated region. In the figure, only candidate points P1 to P4 are illustrated in order to avoid cumbersomeness, but the points are not limited to the four points. Then, as illustrated in FIGS. 5(a) to 5(c), a distance from each of the candidate points P1 to P4 to the tissue boundary 23 is calculated, and a distance from each of the candidate points to the tissue boundary 23 which is the shortest distance is acquired, respectively. Then, a candidate point (P3 in the illustrated example) of a shortest distance d3, that is, the shortest distance from each of the candidate points P1 to P4 is the longest is acquired. That is, the candidate point P3 which is the farthest from the tissue boundary 23 is selected. Then, a profile of a circle having the shortest distance d3 which is the longest as a radius R around the candidate point P3 is set as an ROI 24. The ROI 24 is not limited to a circle and may be a profile of a regular polygon inscribed in the circle, for example. Profile data of the set ROI 24 is outputted to the ROI image generation unit 13. Moreover, if tissue elasticity in the ROI 24 is to be acquired, it is outputted to the elasticity calculation unit 9 at the same time.

The ROI image generation unit 13 generates profile data of the ROI 24 on the basis of the coordinate data of the ROI 24 and outputs it to the display image generation unit 7. The display image generation unit 7 displays the profile of the ROI 24 on the image display unit 8 by superimposing it on the contrast image in accordance with the profile data of the inputted ROI image.

As described above, even if the figure formed by the tissue boundary 23 as illustrated in FIG. 4(a) is not closed, the ROI 24 as wide as possible can be set on the biological tissue 21 in which the examiner is interested. Moreover, the labor of the examiner relating to the ROI setting can be made less, and even if the boundary line of the biological tissue of interest is missing, the ROI can be set reliably. As a result, measurement time can be reduced, and inappropriate setting by manual setting by a person can be eliminated and thus, such an effect that reproducibility of a measured value can be improved is obtained. As a result, since stable clinical data of the nature of the biological tissue in the region of interests can be measured, reliability of statistic data and reliability of diagnosis can be improved.

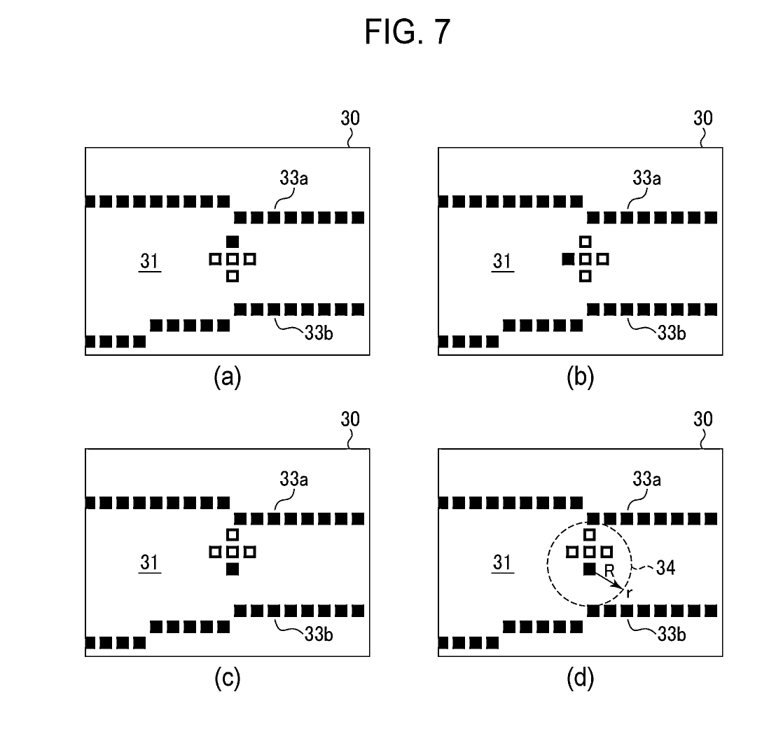

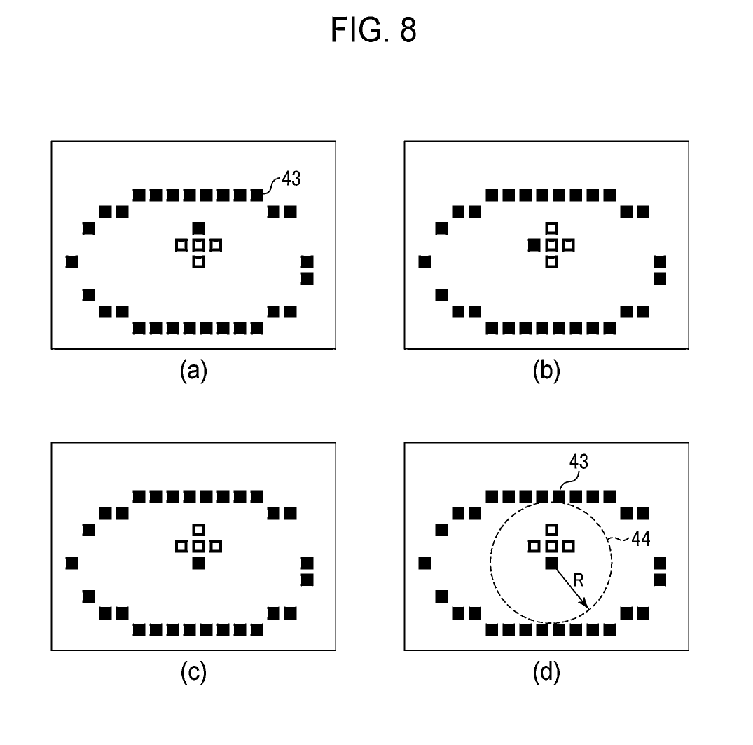

FIG. 7 illustrate operation state diagrams when a circular ROI 34 is automatically set in a notable tissue such as the fat layer 31 in which the tissue boundaries are layered as in the contrast image in FIG. 4(b). Moreover, FIG. 8 illustrate operation state diagrams when a circular ROI 44 is automatically set to a notable tissue 41 in a state in which the figure formed by a tissue boundary 43 is closed as in FIG. 4(c). In those examples, since specific processing procedures are the same as those in the example in FIGS. 5, explanation is omitted. In both FIGS. 7 and 8, centers are set so that peripheral edges in the radial direction of the radiuses R of the ROIs 34 and 44 are in contact with the tissue boundaries 33a and 33b and the tissue boundary 43, but it is needless to say that one of the peripheral edges in the radial direction is not in contact with the tissue boundary depending on the setting of the designated point P0.

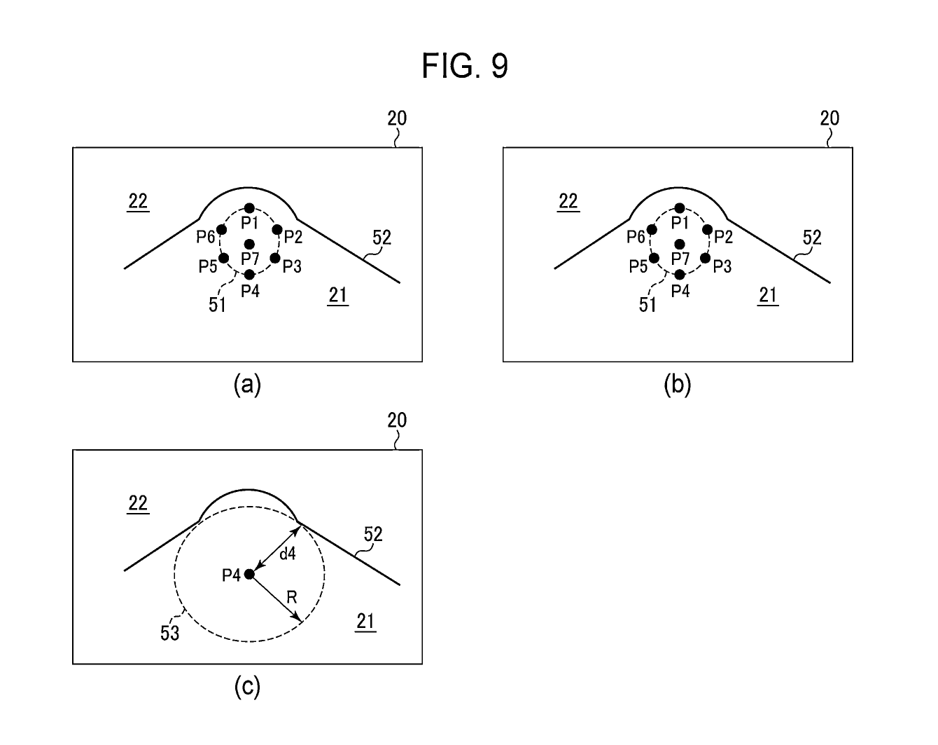

FIG. 9 explain an ROI automatic setting method when a designated region 51 with an arbitrary two-dimensional shape is inputted/set by the examiner through the input device. As illustrated in FIG. 9(a), the examiner paid attention to the biological tissue 21 and inputted/set the oval designated region 51 in the contrast image 20 by drawing. In this case, a plurality of candidate points P1 to P7 are set in an inner region of the designated region 51. Then, speckle removal processing of the contrast image 20 is executed. Then, in the contrast image 20 from which the speckles have been removed, detection processing of a boundary of the biological tissue 21 containing the designated region 51 is executed. For example, the first method of the above-described boundary detection method is applied and explained. Pixel values of the contrast image 20 which is an ultrasound image is subjected to partial differentiation along a search line set radially from an arbitrary reference point in the plurality of candidate points P1 to P7. On the basis of distribution of absolute values of the acquired partial differential values, a pixel at a threshold value or more determined in advance by the partial differential value is acquired and a tissue boundary 52 is detected. That is, if the properties of the adjacent biological tissues are the same, the partial differential values of the pixel values in the search direction are small, and if the properties of the adjacent biological tissues are different, absolute values of the partial differential values of the pixel values in the search direction become large on the boundary. By acquiring this for a plurality of search lines, a so-called peak distribution (ridge) of the absolute values of the partial differential values is obtained, and this is detected as the tissue boundary 52.

The minimum distance calculation portion 124 calculates a distance between the tissue boundary 52 detected as above and each of the candidate points P1 to P7, and the shortest distances whose distance from each of the candidate points to the tissue boundary 52 is the shortest are acquired, respectively. Then, the maximum distance calculation portion 125 acquires a candidate point (P4 in the illustrated example) of the shortest distance d4 whose shortest distance from each of the candidate points P1 to P7 is the longest is acquired. That is, the candidate point P4 which is the farthest from the tissue boundary 52 is selected. Then, a profile of a circle having the longest shortest distance d4 as the radius R around the candidate point P4 is set as an ROI 53. As described above, in the example in FIG. 9, too, similarly to the example in FIG. 5, the ROI 53 as wide as possible can be set for the biological tissue 21 to which the examiner pays attention. As a result, stable clinical data of the properties of the biological tissue in the region of interest can be measured, and thus, reliability of statistical data and reliability of diagnosis can be improved. It is preferable that the search range along the search line in the example in FIG. 9 is limited. According to this, in the case of a figure in which the tissue boundary 52 is not closed, prolongation of calculation time of the distance calculation can be avoided.

Here, a specific example to which the ROI automatic setting method of the present invention is applied will be explained. As described above, in order to measure the properties of a lesion site and to make it contribute to diagnosis, elasticity of the lesion site is measured and diagnosed in practice. As the easiest elasticity data, a strain value of a biological tissue is generally used, but since the strain value depends on stress acting on the biological tissue in measurement, in order to obtain statistical clinical data suitable for diagnosis, elasticity data of the lesion site measured for different individuals needs to be collected as objective clinical data. Thus, conventionally, in an ultrasound image measured under the same stress, by evaluating a size of the strain ε of the biological tissue of the lesion site by a strain ratio (ε/εᵣ) using strain εᵣ of a normal biological tissue (a fat layer, for example) other than a lesion site with less individual difference as a reference, elasticity of the lesion site is objectively evaluated. An elastic-modulus ratio may be used instead of a strain ratio, and both are inclusively referred to as an elastic ratio, but the strain ratio is explained as an example in this embodiment.

Regarding this strain ratio (ε/εᵣ), regions of interest (ROI) are set in a reference part and a lesion site, respectively, and a ratio of a strain average value in each ROI is calculated. Moreover, by setting each ROI widely in a biological tissue having the same elasticity, the number of measurement points (normally, pixels) included in the ROI is increased, so that errors are reduced and stable strain average values are acquired.

Thus, by using the ROI automatic setting method of the present invention, the ROI 24 is set in the biological tissue 21 which is a lesion site of the region of interest in FIG. 4(a), and the elasticity calculation unit 9 acquires a strain average value ε of the biological tissue in a set ROI 21. Moreover, as a reference part, an ROI 34 is set in the fat layer 31 in FIG. 4(b) with less individual difference, and the elasticity calculation unit 9 acquires a strain average value εᵣ of the biological tissue in the set ROI 34. Then, the elasticity calculation unit 9 acquires a strain ratio (ε/εᵣ) obtained by dividing the strain average value ε of the notable tissue by the strain average value εᵣ of the fat layer 31, superimposes and displays the strain ratio on the elasticity image. That is, by acquiring the strain ratio obtained by normalizing or indexation of the strain average value measured under various conditions for the lesion site of each patient by the strain average value εᵣ of the fat layer 31 with less individual difference measured under the same condition, even if the individual difference or measurement conditions are different, objective clinical data can be collected.

Particularly, according to the present invention according to this Embodiment 1, since the region of interest (ROI) can be automatically set as widely as possible, the strain ratio of the biological tissue can be uniformly acquired, and reliability of clinical data relating to elasticity can be improved. The clinical data with high reliability enables accurate diagnosis in individual diagnoses and narrowing of a standard value relating to the diagnosis.

As described above, the region of interest setting method of the present invention according to Embodiment 1 has the first step for setting a plurality of candidate points in an arbitrary designated region designated in a notable tissue in an ultrasound image of an object by an input device, the second step for calculating a change of a pixel value in a two-dimensional direction of the ultrasound image and for detecting a tissue boundary, the third step for acquiring a shortest distance between the detected tissue boundary and each of the candidate points and for setting a circle or a regular polygonal region inscribed in the circle having the shortest distance which is a longest thereof as a radius around the candidate point having the shortest distance which is the longest thereof, and the fourth step for imaging the region of interest which was set and superimposing it on the ultrasound image and for displaying it on an image display unit, and the region of interest as wide as possible can be automatically generated in the notable tissue for measuring the properties of the biological tissue.

In this Embodiment 1, in the first step, a circular region having a radius determined in advance around an arbitrary designated point designated by the input device can be made a designated region. Moreover, in the first step, the two-dimensional region designated by drawing using the input device can be made the designated region.

Moreover, in the second step, by partially differentiating the pixel value in orthogonal two directions of the ultrasound image, the tissue boundary can be detected on the basis of the absolute value of the partial differential value. Furthermore, in the second step, by partially differentiating the pixel value of the ultrasound image along the search line set radially from one of the candidate points, the tissue boundary can be detected on the basis of the absolute value of the partial differential value. Still further, in the second step, the search range of the tissue boundary is preferably set to the maximum range in advance.

Moreover, the ultrasound diagnostic apparatus of Embodiment 1 can be constituted by including an ultrasound image generation portion for transmitting/receiving ultrasonic waves to an object and for generating an ultrasound image on the basis of the received reflected echo signal, the image display unit for displaying the ultrasound image, the input device for setting the designated region by a point or a region in a notable tissue of the ultrasound image displayed on the image display unit, a tissue boundary detection portion for detecting the tissue boundary on the basis of a change in the pixel value in the two-dimensional direction of the ultrasound image, the minimum distance calculation portion for acquiring a shortest distance from each of the candidate points to the tissue boundary, respectively, the maximum distance calculation portion for acquiring a circle having the shortest distance which is the longest around the candidate point with the shortest distance which is the longest thereof as a radius, and the region of interest setting portion for setting the region of a circle or a polygon inscribed with the circle as the region of interest. Moreover, it can be constituted by including a region of interest image generation portion for generating an image of the region of interest and for drawing it by superimposing it on the ultrasound image displayed on the image display unit.

Moreover, the elasticity calculation portion for acquiring a strain value of the biological tissue on the basis of the ultrasound image generated by the ultrasound image generation portion can be provided, the notable tissues set by the input device are a lesion site and a fat layer, the region of interest setting portion sets the regions of interest in the lesion site and the fat layer, respectively, and the elasticity calculation portion can be configured to acquire a strain average value of the region of interest set in the fat layer and a strain average value of the region of interest set in the lesion site and to acquire the strain ratio by dividing the strain average value of the lesion site by the strain average value of the fat layer.

In general, ROI setting is made by an examiner who is a medical technologist or a doctor using the input device such as a pointing device and drawing a circular or a rectangular region on an ultrasound image displayed in the monitor and the ROI can be set by changing the size of the region, for example. However, it is cumbersome to manually set the ROI in conformity to the boundary of a lesion site, and the ROI might be set including the region beyond the boundary of the lesion site depending on the examiner. To the contrary, a narrow ROI might be set so that the boundary of the lesion site is not run over. Thus, the measurement data of the strain average values might be varied among the examiners, and there is a problem with reproducibility of the measurement data. This problem is commonly applied to setting of the ROI for which not only elasticity data but property data of the biological tissue is measured.

On the other hand, as a technology for detecting a boundary of a biological tissue, a technology for detecting a profile line of a boundary of ventricles of the heart is proposed in Japanese Patent No. 4607263, but the technology of this literature is suitable for detection of a profile which becomes a closed figure such as a boundary of a ventricle, but if a part of the boundary of the biological tissue to be diagnosed is obscure such as a lesion site, that is, if the boundary line of the biological tissue to be diagnosed is not a closed figure, for example, the technology cannot be applied to generation of an ROI. Moreover, in a biological tissue such as a fat layer, if the boundary line is layered and does not form a closed figure, the technology in this literature cannot be applied to setting of an ROI, either.

In this point, according to Embodiment 1, as described above, since the region of interest ROI as wide as possible can be automatically set, a strain ratio of a biological tissue can be uniformly acquired, and reliability of clinical data relating to elasticity can be improved. Since clinical data with high reliability enables accurate diagnosis in individual diagnosing and narrowing of standard values relating to the diagnosis, reliability of diagnosis can be improved.

### Embodiment 2

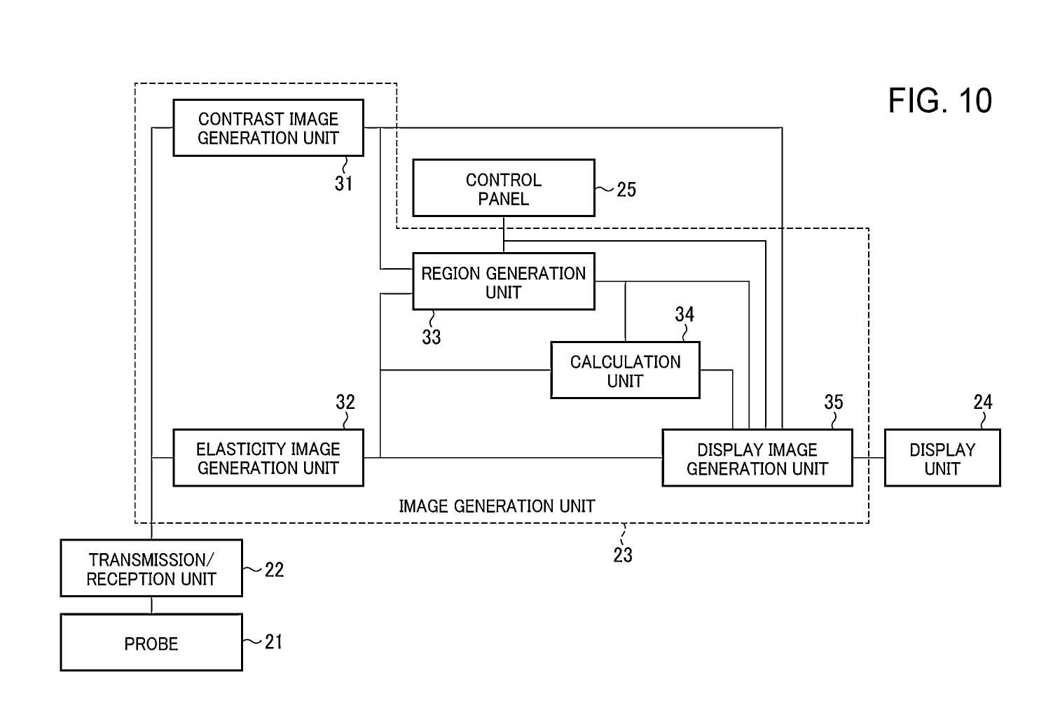

In an ultrasound diagnostic apparatus of Embodiment 2, when one of regions of interest in a comparative relationship is generated, the other region of interest is automatically generated. As a result, measurement of elasticity ratio with less variation can be made possible. Embodiment 2 includes, as illustrated in FIG. 10, a probe 21, a transmission/reception unit 22, an image generation unit 23, and a display unit 24. Each of these portions can be controlled from a control panel 25. On the control panel 25, an arbitrary parameter for generating an ultrasound image is set by an operator. For example, the control panel 25 includes operating devices such as a mouse, a keyboard, a trackball, a touch pen, a joy stick and the like and is configured such that setting of an image display condition and the like can be inputted by using the operating devices.

The probe 21 is formed by disposing a plurality of transducers and transmits ultrasonic waves (acoustic signal) through the transducer to an object brought into contact with that and receives a reflected signal from the object. The transmission/reception unit 22 drives the probe 21 and transmits the ultrasonic waves and also applies signal processing to the reflected signal from the object. In this case, the transmission/reception unit 22 forms a transmission/reception beam, transmits the ultrasonic waves to the object from the probe 21 and gives frame data generated by using the received reflective signal to the image generation unit 23. For example, the transmission/reception unit 22 is constituted by including a transmission circuit, a transmission delay circuit, a reception circuit, a reception delay circuit, a phasing addition circuit and the like. The transmission circuit generates a transmission pulse for generating ultrasonic waves by driving the probe 21, and the transmission delay circuit sets a convergence point of the transmitted ultrasonic waves to a certain depth and repeatedly transmits the ultrasonic waves from the transmission circuit at a time interval to the object through the probe. On the other hand, the reception circuit receives a time-series reflected echo signal generated from the object through the probe 21, and the reception delay circuit takes in the reflective echo signal in accordance with a timing signal inputted from the transmission delay circuit and executes reception processing (generation of RF signal) such as amplification. Moreover, the phasing addition circuit matches the phases of the reflected echo signals taken into the reception delay circuit and adds them up. At that time, the phasing addition circuit executes phase control by inputting an RF signal amplified in the reception delay signal, forms an ultrasound beam to one or a plurality of convergence points and generates RF signal frame data which is ultrasound cross-section region data in a time series.

The image generation unit 23 generates an ultrasound image using the reflected signal subjected to the signal processing by the transmission/reception unit 22 and includes a contrast image generation unit 31, an elasticity image generation unit 32, a region generation unit 33, a calculation unit 34 and a display image generation unit 35.

The contrast image generation unit 31 receives an input of ultrasound cross-section region data of a cross section region of the object or specifically, the RF signal frame data from the phasing addition circuit of the transmission/reception unit 22, executes signal processing such as gain correction, log compression, detection, contour enhancement, filter processing and the like and generates a cross-section region image (a cross-section region image by monochrome contrast brightness (so-called B-mode image), for example). Moreover, the contrast image generation unit 31 includes an A/D converter for converting the cross-section region image data to a digital signal, a frame memory for storing a plurality of pieces of the converted cross-section region image data in a time series, and a monochromic DSC (Digital Scan Converter) constituted by including a controller and the like. The monochromic DSC obtains the cross-section region frame data in the object stored in the frame memory as one image and reads out the obtained cross-section region frame data in TV synchronization.

The elasticity image generation unit 32 acquires a strain and an elastic modulus of a tissue in the cross-section region on the basis of the ultrasound cross-section region data of the cross section region of the object and also generates an elasticity image in the cross section region on the basis of the acquired strain and elastic modulus. In this case, the elasticity image generation unit 32 is constituted by including a frame data obtaining portion, a displacement measurement portion, a pressure measurement portion, a color DSC and the like. That is, the elasticity image generation unit 32 calculates a strain and an elastic modulus of the biological tissue corresponding to each point on the cross-section region image on the basis of displacement information of a biological tissue measured by the displacement measurement portion using the RF signal frame data generated by the phasing addition circuit, that is, a displacement vector, for example, and constitutes an elasticity image signal, that is, the elasticity frame data on the basis of the strain and the elastic modulus. When the strain and the elastic modulus of a biological tissue is to be calculated, the elasticity image generation unit 32 takes into consideration of a pressure value outputted from the pressure measurement portion. Here, strain data is calculated by applying spatial differentiation of a movement amount of the biological tissue, that is, displacement, for example. Moreover, data of the elastic modulus is calculated by dividing a change in the pressure by a change in the strain. For example, assuming that the displacement measured by the displacement measurement portion is L(X) and a pressure measured by the pressure measurement portion is P(X), a strain ΔS(X) can be calculated by applying spatial differentiation to L(X) and thus, it can be acquired by using an equation ΔS(X)=ΔL(X)/ΔX. Moreover, a Young's modulus Ym(X) of the elastic modulus data can be acquired by using an equation Ym=ΔP(X)/ΔS(X). Since the elastic modulus of a biological tissue corresponding to each point on the cross-section region image can be acquired from this Young's modulus Ym, two-dimensional elasticity image data can be continuously obtained. The Young's modulus is a ratio to a simple tension stress applied to an object and a strain generated in parallel with the tension.

Here, in the elasticity image generation unit 32, the frame data obtaining portion obtains from the transmission/reception unit 22 frame data of the reflected echo signal obtained by transmitting ultrasonic waves from the probe 21 by applying pressure to a biological tissue of the object. Moreover, the frame data obtaining portion stores a reflected echo signal group corresponding to a scanned surface (cross-section surface) of an ultrasonic beam in a memory or the like in a plurality of frames together. The displacement measurement portion sequentially takes in a plural pairs of frame data with different obtainment time stored in the frame data obtaining portion and acquires displacement vectors at a plurality of measurement points on the cross section surface on the basis of the taken-in pair of frame data. Then, the elasticity image generation unit 32 applies various types of image processing such as smoothing processing in a coordinate plane, contrast optimization processing, smoothing processing in a time-axis direction between frames and the like to the frame data of each elasticity information constituted by the frame data obtaining portion and sends it the color DSC. The color DSC converts the elasticity frame data so as to match display of the display unit 24. That is, the color DSC has a function of giving hue information to the elasticity frame data and converts the data to image data added with red (R), green (G), and blue (B) which are three primary colors of light on the basis of the elasticity frame data. For example, the color DSC converts the elasticity data with large strain to a red code and converts the elasticity data with small strain to a blue code.

The display image generation unit 35 is constituted by including a frame memory, an image processing portion, an image selection portion and the like and generates a synthetic image or a parallel image of the cross-section region image and the elasticity image by a method represented by α blending. The frame memory stores the cross-section region image data from the monochromic DSC of the contrast image generation unit 31 and the elasticity image data from the color DSC of the elasticity image generation unit 32. Moreover, the image processing portion synthesizes the cross-section region image data and the elasticity image data stored in the frame memory by changing a synthesizing ratio. Brightness information and hue information of each pixel of the synthetic image become the one in which each information of the monochrome ultrasound sonogram and the color elasticity image at the synthesizing ratio. Moreover, the image selection portion selects an image to be displayed from the cross-section region image data and the elasticity image data in the frame memory and the synthetic image data in the image procession portion and has the selection displayed on the display unit 24.

The display unit 24 displays images such as the cross-section region image selected by the image selection portion of the display image generation unit 35 and the elasticity image and the like and a first diagnosis region and a second diagnosis region generated by the region generation unit 33, which will be described later, together with the elasticity ratio between the first diagnosis region and the second diagnosis region calculated by the calculation unit 34 to be visually recognizable.

In this embodiment, the image generation unit 23 includes the region generation unit 33 and the calculation unit 34 in addition to the above-described contrast image generation unit 31, the elasticity image generation unit 32, and the display image generation unit 35. The configurations of the region generation unit 33 and the calculation unit 34 which are feature portions of the present invention will be explained below.

The image generation unit 23 generates the first diagnosis region and the second diagnosis region as two regions (regions of interest) to be offered for diagnosis. Specifically, in the region generation unit 33, the first diagnosis region and the second diagnosis region are generated. In this case, a first reference position included in the first diagnosis region of the ultrasound image displayed on the display unit 24 is set by an operator from the control panel 25. Then, the region generation unit 33 generates the first diagnosis region in a region including the first reference position set on the ultrasound image. Moreover, the region generation unit 33 generates the second diagnosis region to be generated on the ultrasound image by using positional information of the first diagnosis region, protrusion to an outside of the ultrasound image, and edges and peripheral tissues of the first diagnosis region. In this case, it is only necessary that the region generation unit 33 generate the second diagnosis region by using a range not including the first diagnosis region, a range from which the second diagnosis region to be generated on the ultrasound image does not protrude, and a range in which the second diagnosis region is not provided on the edges or the peripheral tissues of the first diagnosis region. In this embodiment, the region generation unit 33 generates the second diagnosis region by further using a range in which the second diagnosis region is not provided at a position with a depth larger than that of the first reference position and a straight line passing through the first reference position, respectively.

Here, in this embodiment, such a case is assumed that the first reference position is set in a disease site of the displayed ultrasound image, and the second diagnosis region is generated in a reference part of the disease site. Specifically, a case in which the first reference position (that is, the first diagnosis region including the first reference position) is set in a tumor site as an example of the disease site, and the second diagnosis region is set in a fat part as an example of the reference part of the disease site is assumed. Hereinafter, the first diagnosis region is referred to as a tumor ROI and the second diagnosis region as a fat ROI. However, these diagnosis regions can be set in arbitrary parts and are not particularly limited to the tumor site or the fat part. Moreover, in this embodiment, the region generation unit 33 generates a circular tumor ROI having a first radius from the first reference position with the first reference position as a center of the tumor ROI which is the first diagnosis region and generates a circular fat ROI having a second radius from the second reference position with the second reference position as a center of the fat ROI which is the second diagnosis region. In this case, a radius value of the tumor ROI is calculated by the region generation unit 33 on the basis of the first reference position, and a radius value of the fat ROI is held as a specified value in advance by the region generation unit 33. That is, the region generation unit 33 holds the radius of the fat ROI which is the second diagnosis region as the specified value in advance and sets the held specified value to a radius of the fat ROI (second radius). However, the shapes of these ROIs are not particularly limited and can be polygonal shapes such as elliptic, triangular, square or the like, and two ROIs may have different shapes. In addition, in this embodiment, a case in which two regions are generated in the region generation unit 33 is explained as an example, but a case in which three or more regions are generated in the region generation unit 33 can also be assumed.

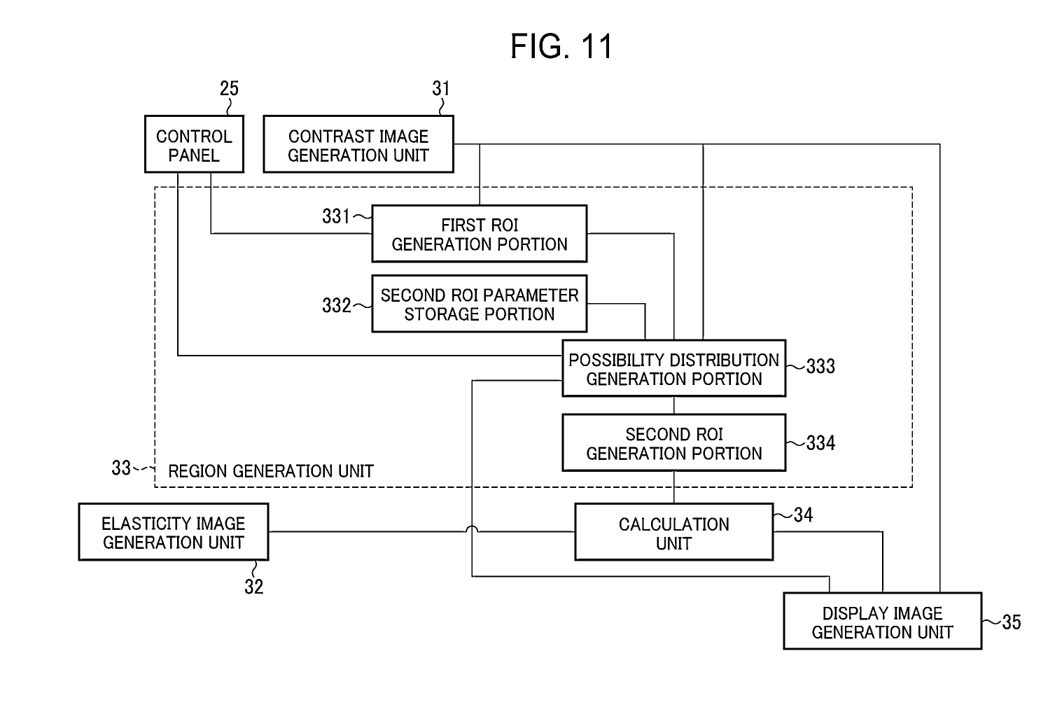

FIG. 11 is a block diagram exemplifying a configuration of the region generation unit 33 of this embodiment. As illustrated in FIG. 11, the region generation unit 33 includes a first ROI generation portion 331, a second ROI parameter storage portion 332, a possibility distribution generation portion 333, and a second ROI generation portion 334.

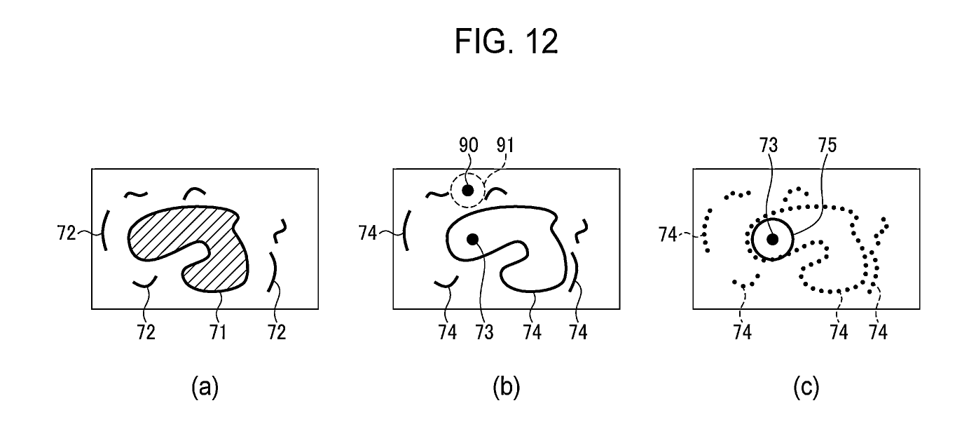

The first ROI generation portion 331 calculates a center and a radius of a circle substantially inscribed in a tumor edge from a center position of the ultrasound image and the tumor site so as to set the tumor ROI and gives the center position and the radius value of the tumor ROI to the possibility distribution generation portion 333. FIG. 12 schematically illustrate a setting procedure of the tumor ROI in the first ROI generation portion 331. In setting the tumor ROI which is the first diagnosis part, the first ROI generation portion 331 takes in the center position of the tumor site as a reference position of the tumor ROI. In this case, the center position of the tumor site is set by the operator from the control panel 25. At that time, the operator operates the control panel 25 by using the operating device so as to display the ultrasound image on the display unit 24 and sets the center position of the tumor site on such image. FIG. 12(a) illustrates a cross-section region image of the tumor site by monochrome contrast brightness generated by the contrast image generation unit 31 as an example of an ultrasound image to be displayed in which a shaded part is a tumor tissue part 71 and a part indicated by a solid line in the periphery of such tumor tissue part 71 is a peripheral tissue part 72 such as a ligament and the like. In this figure, the tumor tissue part 71 is indicated by shading for convenience, but on the display screen of the display unit 24, it is indicated by monochrome contrast brightness. It is also possible to set the center position of the tumor site on the elasticity image generated by the elasticity image generation unit 32. In this case, it is only necessary for the operator to set an arbitrary position assumed to be a center of the tumor tissue part 71 as the center position of the tumor site. FIG. 12(b) illustrates an example of a center position 73 of the tumor site set as above. In FIG. 12(b), a gradient lengths 74 of the tumor tissue part 71 and the peripheral tissue part 72, and a fat ROI 91 and its center position 90 which is the second diagnosis region, which will be described later, are also illustrated. The gradient lengths 74 of the tumor tissue part 71 and the peripheral tissue part 72 are distribution of values which can be calculated with absolute values of partial differentiation of the image brightness and can be acquired by square-root of sum of squares of a partial differential value in each direction by convolving a Sobel operator known in an edge extraction program in an image, calculating partial differential values in a horizontal direction and a vertical direction, for example. FIG. 12(c) indicates ridges (ridge line parts) of the gradient lengths 74 acquired as above by dotted lines.

Then, as illustrated in FIG. 12(c), the first ROI generation portion 331 generates a circle having the shortest distance in distances from the center position 73 of the tumor site set by the operator to the ridge of the gradient length 74 as a radius (first radius) to a tumor ROI 75. In FIG. 12(c), the tumor ROI 75 is indicated by a solid line, and the ridges of the gradient lengths 74 are indicated by the dotted lines. The ridge of the gradient length 74 is a spot with a protrusion when seen in a gradient direction, and when a value of a gradient length pixel in the gradient direction at each of gradient length pixel position and a value of the gradient length pixel in an anti-gradient direction are compared, and if the gradient length pixel of interest has the longest value, it is approved to correspond to the ridge (ridge line part). As a result, a radius (the shortest distance from the center position 73 to the ridge of the gradient length 74) of the tumor ROI 75 can be acquired as the first radius. That is, only by setting by the operator the center position 73 of the tumor site as the reference position (first reference position), the tumor ROI 75 (first diagnosis region) including such reference position can be automatically generated.

The possibility distribution generation portion 333 generates positional information of the second reference position for automatically setting the fat ROI which is the second diagnosis part to the fat part, that is, positional information (hereinafter referred to as possibility distribution) indicating whether it is a position that can be set as the center position of the fat ROI or not. In this embodiment, the possibility distribution generation portion 333 generates possibility distribution indicating a position that can be a center position of the fat ROI on the basis of the center position 73 and the radius value of the tumor ROI 75 substantially inscribed in the tumor site given by the first ROI generation portion 331, the radius value which is a parameter of the fat ROI held as the specified value in advance in the second ROI parameter storage portion 332, and the ultrasound image (the cross-section region image generated by the contrast image generation unit 31 and the elasticity image generated by the elasticity image generation unit 32) and gives the result to the second ROI generation portion 334. In this embodiment, since the fat ROI is set as a circular shape having the reference position (second reference position) as the center position, the second ROI parameter storage portion 332 holds the radius value (second radius) of the fat ROI as the specified value, and such radius value is taken in by the possibility distribution generation portion 333. That is, the second ROI parameter storage portion 332 stores parameters in advance according to the shape of the fat ROI to be generated. For example, if the fat ROI is to be generated as a triangle including a reference position (second reference position), it is only necessary that a length of one side (a side which becomes a reference) from the reference position and an inclination angle to the reference side and the like are held as specified values. Moreover, if the fat ROI is to be generated as a rectangle including the reference position (second reference position), for example, it is only necessary that distances in an X-direction and in a Y-direction crossing each other with respect to the reference position are held as specified values, respectively.

The possibility distribution is generated by the possibility distribution generation portion 333 on the basis of a predetermined condition, but at that time, the possibility distribution is generated under a plurality of conditions according to the tumor ROI 75 which is the first diagnosis region. As such conditions, a value (hereinafter referred to as a characteristic value) indicating whether or not the reference position (second reference position) of the fat ROI can be set on the ultrasound image is given for each position on a plurality of ultrasound images and the possibility distribution is generated by calculation using the characteristic value at the same position on the plurality of ultrasound images.

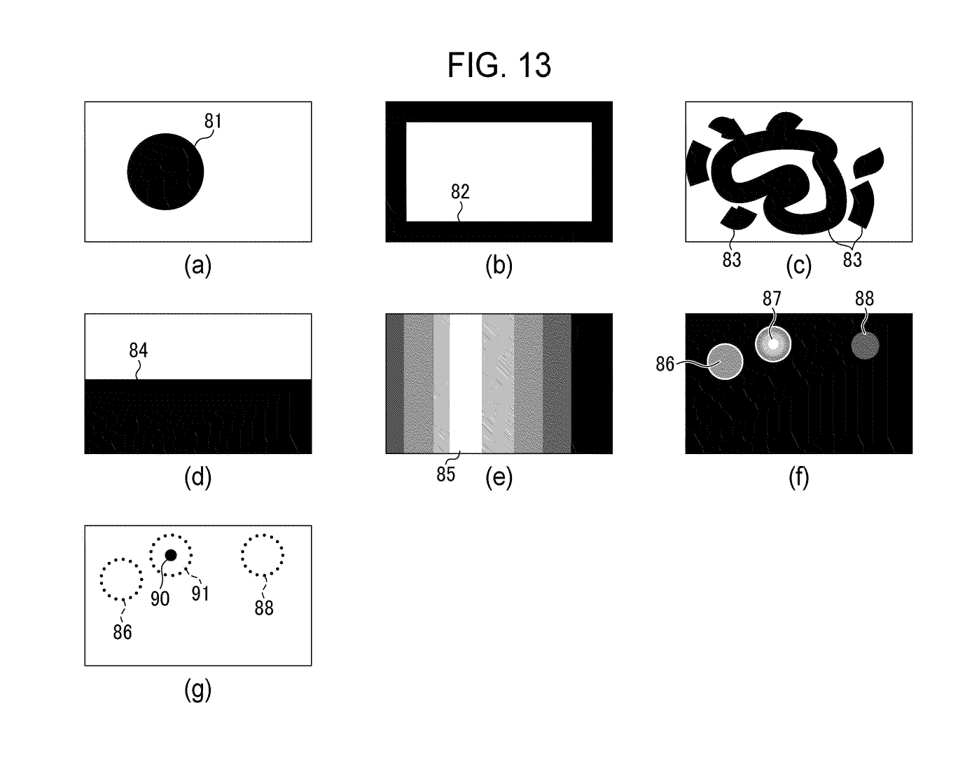

FIG. 13 schematically illustrate conditions for generating the possibility distribution in the possibility distribution generation portion 333 and the generated possibility distribution and the fat ROI which is the second diagnosis region generated by using the possibility distribution. Such conditions indicate whether the center position of the fat ROI can be set on the ultrasound image or not, and individual condition examples are illustrated in FIGS. 13(a) to 13(e). In FIGS. 13(a) to 13(e), a position with no possibility that the center position of the fat ROI is set (more specifically, a pixel) is indicated in black, a position with high possibility in white, and positions with some possibility but not so high in gray contrast according to the possibility for discriminating each position. At that time, as the characteristic value, 0 is given to the black position and 1 to the white position for each pixel. For gray positions, the characteristic value larger than 0 and smaller than 1 is given so that the value becomes larger as its darkness increases.

FIG. 13(a) is a condition view using the tumor ROI 75 which is the first diagnosis region generated by the first ROI generation portion 331 (FIG. 12(c)) or specifically, a range not including the tumor ROI 75. That is, FIG. 13(a) illustrates a condition of the center position (second reference position) of the fat ROI without the tumor ROI 75 (FIG. 12(c)) superimposed on the fat ROI. In this case, a region 81 indicated by a black circle is a circular region having a value obtained by adding the radius value of the fat ROI (hereinafter referred to as a fat ROI radius) given by the second ROI parameter storage portion 332 to the radius of the tumor ROI 75 as a radius around the center position 73 of the tumor ROI 75 (FIG. 12(c)). That is, according to FIG. 13(a), if the center position of the fat ROI is set in the region 81, the fat ROI is superimposed on the tumor ROI 75, but by setting the center position to a region other than the region 81, it is known that the fat ROI is positioned separately from the tumor ROI 75 without superimposing on it.

FIG. 13(b) is a condition view using a protrusion to the outside of the ultrasound image or specifically, a range from which the fat ROI to be generated on the ultrasound image does not protrude. That is, FIG. 13(b) illustrates a condition that the fat ROI is prevented from protruding from the display region of the ultrasound image in the display unit 24. In this case, a region 82 indicated by a black frame is a frame region having the fat ROI radius as a width. That is, according to FIG. 13(b), if the center position of the fat ROI is set in the region 82, the fat ROI protrudes from the display region, but by setting the center position in a region other than the region 82, it is known that the fat ROI is fully accommodated in the display region without protruding from the display region.

FIG. 13(c) is a condition view using positional information of the edges or peripheral tissues of the tumor ROI 75 (FIG. 12(c)) which is the first diagnosis region or specifically, a range in which the fat ROI is not provided on the edge or peripheral tissue of the tumor ROI 75. That is, FIG. 13(c) illustrates a condition that the fat ROI is not positioned on the edge of the tumor tissue part 71 or the peripheral tissue part 72 such as a ligament (FIG. 12(a)). The edge of the tumor tissue part 71 or the peripheral tissue part 72 such as a ligament corresponds to a region displayed with high brightness on a cross-section region image by monochrome contrast brightness generated by the contrast image generation unit 31, that is, the ridges (ridge line part) of the gradient lengths 74 of the tumor tissue part 71 and the peripheral tissue part 72, for example. Moreover, the edge of the tumor tissue part 71 or the peripheral tissue part 72 such as a ligament corresponds to a region indicated as high hardness in the elasticity image generated by the elasticity image generation unit 32, for example. In this case, a region 83 indicated in black illustrates a region with thickness of the fat ROI radius from the edge of the tumor tissue part 71 or the peripheral tissue part 72 such as a ligament (high brightness region of the cross-section region image and a high hardness region of the elasticity image) by black lines. At that time, it is only necessary that a logical product of the high brightness region of the cross-section region image and the high hardness region of the elasticity image is calculated, and convolution calculation is made using a disk filled in with the fat ROI radius as a kernel. According to FIG. 13(c), if the center position of the fat ROI is set in the region 83, the fat ROI is positioned on the edge of the tumor tissue part 71 or the peripheral tissue part 72 such as a ligament, but by setting the center position in a region other than the region 83, it is known that the fat ROI is not positioned on the edge of the tumor tissue part 71 or the peripheral tissue part 72 such as a ligament but is positioned separately from the tumor tissue part 71 or the peripheral tissue part 72.

It is only necessary that the possibility distribution generation portion 333 generates the possibility distribution on the basis of the conditions illustrated in FIGS. 13(a) to 13(c). However, by further adding conditions to these conditions, generation accuracy of the fat ROI (in other words, setting accuracy of the center position of the fat ROI) can be improved. Thus, in this embodiment, the conditions illustrated in FIGS. 13(d) and 13(e) are further added in generating the possibility distribution.

FIG. 13(d) is a condition view using a range in which the fat ROI is not provided at a position deeper than the center position 73 (FIG. 12(c)) of the tumor ROI 75 which is the first reference position. That is, FIG. 13(d) illustrates a condition that the center position of the fat ROI is not positioned below the center position 73 of the tumor ROI 75 (a position with a larger depth from the surface of the object). In this case, a region 84 indicated by a black band is a band region located below the center position 73 of the tumor ROI 75. That is, according to FIG. 13(d), if the center position of the fat ROI is set in the region 84, such center position is positioned below the center position 73 of the tumor ROI 75, but by setting the center position to a region other than the region 84, it is known that the center position of the fat ROI is not positioned below the center position 73 of the tumor ROI 75 but is positioned above the center position 73. The condition as illustrated in FIG. 13(d) is used because the fat part is present at a position closer to the body surface of the object than the tumor site in general.

FIG. 13(e) is a condition view using a straight line passing through the center position 73 (FIG. 12(c)) of the tumor ROI 75 which is the first reference position. As an example, FIG. 13(e) indicates a condition under which the center position of the fat ROI can be easily positioned on the straight line passing through the center position 73 of the tumor ROI 75. In this case, the vicinity of the center line of the tumor ROI 75 is a region 85 indicated by a white color, and gradation which becomes gradually gray to black is made as it goes away from the white region 85 toward both left and right sides. That is, according to FIG. 13(e), it is known that possibility that the center position of the fat ROI is positioned is higher at a position closer to the center line of the tumor ROI 75 and it gradually lowers as it goes away from the center line, or in other words, the center position of the fat ROI is preferably positioned at a position closer to the center line of the tumor ROI 75.

Then, the possibility distribution generation portion 333 generates possibility distribution on the basis of the condition as indicated in the above-described condition views illustrated in FIGS. 13(a) to 13(e). In generating the possibility distribution, the possibility distribution generation portion 333 makes calculation using characteristic values of pixels on the same position in the condition views 13(a) to 13(e). In FIG. 13(f), a result obtained by mutually multiplying the characteristic values of the pixels on the same position in the condition views illustrated in FIGS. 13(a) to 13(e) and by performing convolution using the disk of the fat ROI radius as a kernel. Therefore, in the condition views illustrated in FIGS. 13(a) to 13(e), if there is any single point at which it is not likely that the center position of the fat ROI is set at all (a pixel with a characteristic value indicated in black at 0), the pixel is indicated as a point (black pixel) with no possibility that the center position of the fat ROI is set in FIG. 13(f). As illustrated in FIG. 13(f), in this case, only three circular regions 86, 87, and 88 are calculated as regions with possibility that the center position of the fat ROI is set. That is, the possibility distribution generation portion 333 generates an image (FIG. 13(f)) illustrating these regions 86, 87, and 88 as the possibility distribution as the calculation result of such characteristic values. The condition views illustrated in FIGS. 13(a) to 13(e) and the possibility distribution illustrated in FIG. 13(f) do not necessarily have to be displayed on the display unit 24 but may be displayed. If they are to be displayed, the possibility distribution generation portion 333 has such image displayed on the display unit 24 through the display image generation unit 35.

When a value of the reference position of the fat ROI (second reference position of the second diagnosis region) (specifically, the above-described characteristic value) is given to a plurality of ultrasound images (as an example, the condition views illustrated in FIGS. 13(a) to 13(e)), the second ROI generation portion 334 generates a fat ROI by using the characteristic values for the same position of these plurality of ultrasound images. In this embodiment, the second ROI generation portion 334 determines a position (pixel) where a multiplied value of the above-described characteristic value shows the largest value by calculation using the possibility distribution taken in from the possibility distribution generation portion 333 (FIG. 13(f)) and generates the fat ROI using this position as the reference position and also gives it to the calculation unit 34 as the center position of the fat ROI. In this case, the second ROI generation portion 334 selects the region 87 in which the white region is the largest (corresponding to the region where the total of the multiplied values of the above-described characteristic values in the region becomes the largest) in the three regions 86, 87, and 88 illustrated in the possibility distribution (FIG. 13(f)) as a region with the highest possibility that the center position of the fat ROI is set. Then, the second ROI generation portion 334 determines the point (pixel) with the highest value in the multiplied values of the above-described characteristic values in the selected region 87 as the second reference position of the second diagnosis region, that is, the center position of the fat ROI (a black point 90 illustrated in FIG. 13(g)). Moreover, the second ROI generation portion 334 draws a circle having the fat ROI radius (a radius value of the fat ROI given by the second ROI parameter storage portion 332) as a radius around the determined center position. That is, as illustrated in FIG. 13(g), the center position 90 is made a reference position (second reference position), and a circular fat ROI 91 including such reference position is generated. FIG. 13(g) indicates a region selected by the second ROI generation portion 334 (that is, the fat ROI) 91 by a broken line, and indicates the center position 90 of the fat ROI 91 by a black point. Then, using the center position 90 as the reference position, the fat ROI 91 including such center position 90 is displayed on the display unit 24 through the display image generation unit 35 by the second ROI generation portion 334. FIG. 13(g) also illustrates the regions 86 and 88 indicated by the possibility distribution with the fat ROI 91 but display of these regions 86 and 88 may be omitted. As a result, the fat ROI 91 (second diagnosis region) can be automatically generated. That is, only by setting by the operator the center position 73 of the tumor site as the reference position (first reference position), the tumor ROI 75 (first diagnosis region) and the fat ROI 91 (second diagnosis region) can be both automatically generated.

Moreover, in this embodiment, the image generation unit 23 calculates a ratio between a measured value of image data of the ultrasound image representing the first diagnosis region and a measured value of the image data of the ultrasound image representing the second diagnosis region by the calculation unit 34 and displays the calculated ratio on the display unit 24. Specifically, in the calculation unit 34, a ratio between the measured value of the image data representing the tumor ROI 75 and the measured value of the image data representing the fat ROI 91 is calculated, and the calculated ratio is displayed on the display unit 24. In this case, the calculation unit 34 calculates such ratio on the basis of a statistic value including at least one of an average value, a median value, a mode value, a maximum value, and a minimum value of the ultrasound image data. In this embodiment, such a case is assumed as an example that elasticity image data (specifically, elastic modulus data on each point on the image) is used as image data, and an average value of such elastic modulus data is used as a measured value. Therefore, the calculation unit 34 calculates a value obtained by dividing the average value of the elastic modulus data of the tumor ROI 75 in the elasticity image generated by the elasticity image generation unit 32 by the average value of the elastic modulus data of the fat ROI 91 as the elasticity ratio. Then, the calculation unit 34 gives the calculated elasticity ratio between the tumor ROI 75 and the fat ROI 91 to the display image generation unit 35 and has it superimposed on the cross-section region image and an elasticity image and displayed on the display unit 24. That is, the calculated elasticity ratio between the tumor ROI 75 and the fat ROI 91 can be displayed on the display unit 24 with the cross-section region images of the tumor ROI 75 and the fat ROI 91 generated by the contrast image generation unit 31 and the elasticity images of the tumor ROI 75 and the fat ROI 91 generated by the elasticity image generation unit 32.

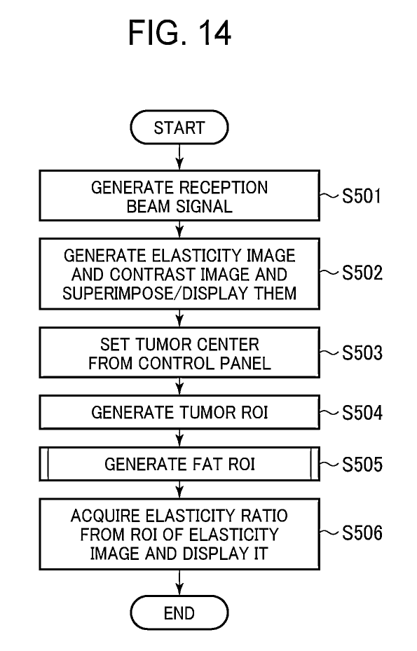

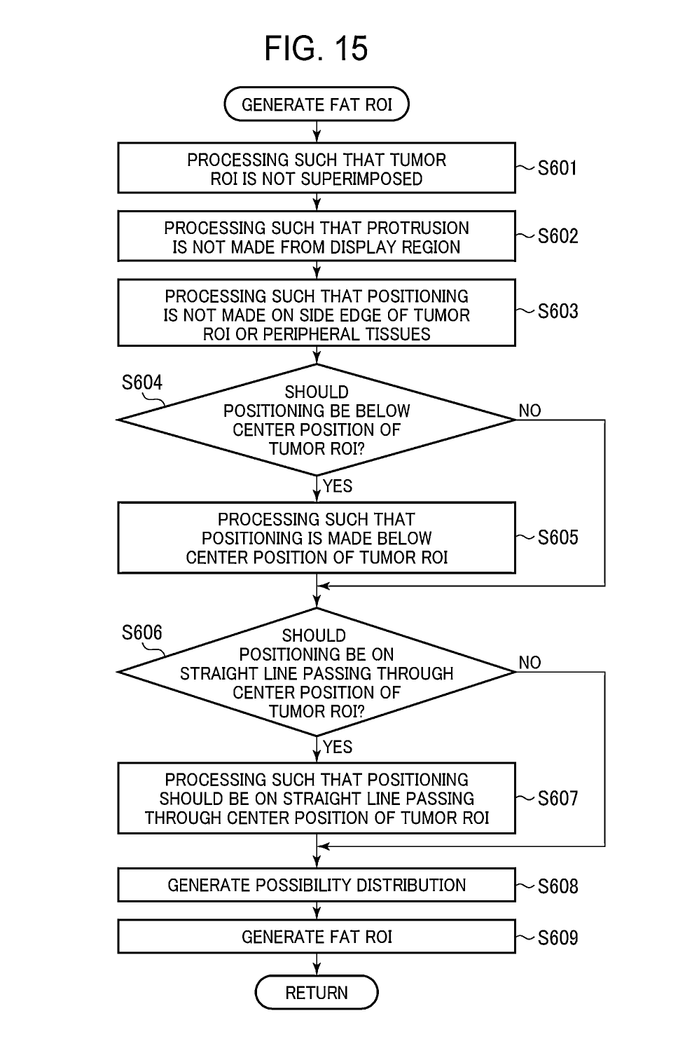

Here, a processing procedure in the ultrasound diagnostic apparatus according to this embodiment with such configuration will be explained by referring to FIGS. 14 and 15. FIG. 14 is a flowchart illustrating an outline of such processing procedure, and FIG. 15 is a flowchart illustrating an example of the procedure for generating the fat ROI. As illustrated in FIG. 14, in such ultrasound diagnostic apparatus, first, while the operator brings the probe 21 into contact with an object, an electric signal (transmission pulse) forming an ultrasound beam from the transmission/reception unit 22 is given to the probe 21. Then, the ultrasound beam is transmitted/received to/from the object through the probe 21, the received ultrasound signal (reflected echo signal) is given to the transmission/reception unit 22, and the reception beam signal (RF signal frame data) is generated in the transmission/reception unit 22 (S501 illustrated in FIG. 14).

The reception beam signal generated in the transmission/reception unit 22 is taken into the contrast image generation unit 31 and the elasticity image generation unit 32 which are the image generation portions 23, a contrast image (as an example, a cross-section region image by monochrome contrast brightness) is generated in the contrast image generation unit 31 and an elasticity image (as an example, a color elasticity image expressed by gradation of hue) is penetrated in the elasticity image generation unit 32. Then, the generated contrast image and elasticity image are taken into the display image generation unit 35, superimposed (synthesized), and displayed on the display unit 24 (S502 illustrated in FIG. 14).

The operator operates the control panel 25 using the operating device and sets the center position of the tumor site on the ultrasound image (as an example, a contrast image) displayed on the display unit 24. For example, by using a position designating device such as a mouse, a touch pen and the like on the control panel 25, the center position of the tumor site is set and then, the set center position is displayed on the ultrasound image by pressing on a display start button or the like (S503 illustrated in FIG. 14). Moreover, the control panel 25 can receive input of a parameter for arbitrarily generating an ultrasound image by the operator.

When the center position of the tumor site is set by the operator, the region generation unit 33 generates the tumor ROI 75 and the fat ROI 91 and has them displayed on the display unit 24 through the calculation unit 34 and the display image generation unit 35. Specifically, on the basis of the ultrasound image (as an example, a contrast image) and the center position of the tumor site, the center position 73 of the first diagnosing region and the radius value are calculated in the first ROI generation portion 331 and the tumor ROI 75 is generated (S504 illustrated in FIG. 14). Moreover, on the basis of the generated tumor ROI 75, the fat ROI 91 is generated (S505 illustrated in FIG. 14). At that time, possibility distribution is generated in the possibility distribution generation portion 333 by the tumor ROI 75 (center position 73 and the radius value) and the ultrasound image (the cross-section region image generated in the contrast image generation unit 31 and the elasticity image generated in the elasticity image generation unit 32). Then, by using the generated possibility distribution, the center position 90 of the second diagnosis region is calculated in the second ROI generation portion 334 and the fat ROI 91 is generated.

As illustrated in FIG. 15, the possibility distribution generation portion 333 gives a characteristic value with a setting condition of the center position of the fat ROI that the fat ROI is not superimposed on the tumor ROI 75 and generates a condition view (FIG. 13(a)) (S601 illustrated in FIG. 15). Moreover, the possibility distribution generation portion 333 gives a characteristic value with the setting condition of the center position of the fat ROI that the fat ROI is not protruded from the display region of the ultrasound image on the display unit 24 and generates a condition view (FIG. 13(b)) (S602 illustrated in FIG. 15). Then, the possibility distribution generation portion 333 gives a characteristic value with the setting condition of the center position of the fat ROI that the fat ROI is not positioned on the side edge of the tumor tissue part 71 or the peripheral tissue part 72 such as a ligament and generates a condition view (FIG. 13(c)) (S603 illustrated in FIG. 15).

Subsequently, it is determined whether or not a condition that the center position of the fat ROI is not positioned below the center position 73 of the tumor ROI 75 (a position with a larger depth from the surface of the object) is adopted to be a setting condition of the center position of the fat ROI (S604). If it is adopted as the setting condition as the result of determination, the possibility distribution generation portion 333 gives a characteristic value with the condition that the center position of the fat ROI is not positioned below the center position 73 of the tumor ROI 75 (condition on the depth) and generates a condition view (FIG. 13(d)) (S605 illustrated in FIG. 15). On the other hand, if it is not adopted as the setting condition as the result of determination, a characteristic value as a condition on the depth is not given and the condition view (FIG. 13(d)) is not generated. Such determination may be made by giving parameters inputted by the operator from the control panel 25 to the possibility distribution generation portion 333.

Moreover, it is determined whether or not a condition that the center position of the fat ROI is positioned on a straight line passing through the center position 73 of the tumor ROI 75 more easily is adopted as a setting condition of the center position of the fat ROI (S606). If it is adopted as the setting condition as the result of determination, the possibility distribution generation portion 333 gives a characteristic value with the condition that the center position of the fat ROI is positioned on a straight line passing through the center position 73 of the tumor ROI 75 more easily (condition on the centerline) and generates a condition view (FIG. 13(e)) (S607 illustrated in FIG. 15). On the other hand, if it is not adopted as the setting condition as the result of determination, a characteristic value as a condition on the centerline is not given and the condition view (FIG. 13(e)) is not generated. Such determination may be made by giving parameters inputted by the operator from the control panel 25 to the possibility distribution generation portion 333.

Then, the possibility distribution generation portion 333 generates the possibility distribution (FIG. 13(f)) on the basis of the conditions obtained by the processing by the above-described S601 to S607 (S608 illustrated in FIG. 15). Specifically, the characteristic values of the pixels at the same position in the condition view obtained by the processing by the above-described S601 to S607 are mutually multiplied and subjected to convolution with the disk of the fat ROI radius as a kernel so as to generate the possibility distribution.

When the possibility distribution (FIG. 13(f)) is generated by the possibility distribution generation portion 333 as above, the fat ROI is generated by using such possibility distribution (609 illustrated in FIG. 15). Specifically, the second ROI generation portion 334 calculates a position (pixel) at which the multiplied value of the characteristic value obtained by the above-described S607 indicates the largest value by the possibility distribution taken in from the possibility distribution generation portion 333, and such position is set to the center position of the fat ROI (the black point 90 in FIG. 13(g)). Moreover, the second ROI generation portion 334 generates the fat ROI 91 having the fat ROI radius (a radius value of the fat ROI given by the second ROI parameter storage portion 332) as a radius around the set center position. The display image generation unit 35 superimposes the ultrasound image and further superimposes the tumor ROI 75 and the fat ROI 91 on such superimposed image and generates a display image. Then, the display unit 24 displays such display image.

Moreover, the calculation unit 34 calculates a value obtained by dividing an average value of the elastic modulus data of the tumor ROI 75 in the elasticity image generated by the elasticity image generation unit 32 by an average value of the elastic modulus data of the fat ROI 91 as an elasticity ratio and gives it to the display image generation unit 35. In the display image generation unit 35, the taken-in value of the elasticity ratio is superimposed on the display image and the display image including such elasticity ratio is generated. Then, such display image is displayed by the display unit 24 (S506 in FIG. 14).

As described above, according to the ultrasound diagnostic apparatus according to Embodiment 2, only by setting by the operator the center position 73 of the tumor site as the reference position (first reference position), the tumor ROI 75 (first diagnosis region) and the fat ROI 91 (second diagnosis region) can be both automatically generated. In short, the two diagnosis regions used for calculation of the elasticity ratio can be semi-automatically generated. Therefore, the elasticity values (elastic modulus) in the two diagnosis regions (the tumor ROI 75 and the fat ROI 91) are not varied, and as a result, accuracy of the calculated elasticity ratio can be improved. As a result, the elasticity ratio with less variation can be displayed. As a result, benignity or malignancy of a tumor, necessity of a surgery and the like, for example, can be accurately determined.

In Embodiment 2, in the image generation unit 23 (the contrast image generation unit 31 and the elasticity image generation unit 32), a contrast image (as an example, a cross-section region image by monochrome contrast brightness) and an elasticity image (as an example, a color elasticity image expressed by gradation of hue) or a superimposed image of them are generated as the ultrasound image, but the ultrasound image to be generated is not limited to them. That is, a type of such ultrasound image does not particularly matter as long as it is any one of brightness, elasticity, strain, a blood flow speed, and a tissue speed. For example, by setting by the operator the reference position (first reference position) to a blood vessel part, the blood vessel part is generated as the first diagnosis region and the fat part as the second diagnosis region semi-automatically, and a measured value ratio of these diagnosis regions (as an example, a ratio of elastic modulus) can be also displayed with a cross-section region image, an elasticity image, and a blood flow image. Alternatively, by generating two diagnosis regions with different tissue speeds, the measured value ratio of these diagnosis regions (as an example, an elastic modulus) can be also displayed with a cross-section region image, an elasticity image, and a tissue speed image (so-called M-mode image).

Moreover, the present invention is not limited to the above-described embodiments but is capable of change/variation within a range described in claims.

The ultrasound diagnostic apparatus of the present invention includes a probe for transmitting an ultrasonic wave to an object and for receiving a reflected signal from the object, a transmission/reception portion for transmitting an ultrasonic wave by driving the probe and for executing signal processing of the reflected signal, an image generation portion for generating an ultrasound image by using the reflected signal subjected to the signal processing, a display unit for displaying the ultrasound image, and a control panel with which an arbitrary parameter is set by an operator for generating the ultrasound image, in which a first reference position included in a first diagnosis region of the displayed ultrasound image is set by the control panel, and the image generation portion is provided with a region generation portion for generating a second diagnosis region to be generated on the ultrasound image by using positional information of the first diagnosis region, protrusion of the ultrasound image to an outside and edges and peripheral tissues of the first diagnosis region.

According to this configuration, two diagnosis regions to be offered for diagnosis of an object can be generated semi-automatically. At that time, possibility distribution (distribution indicating whether or not the position is where the second reference position of the second diagnosis region can be set) can be generated by the positional information of the first diagnosis region, protrusion to an outside of an ultrasound image, and the edges and the peripheral tissues of the first diagnosis region, and the second diagnosis region can be generated by using such possibility distribution.

In the ultrasound diagnostic apparatus of the present invention, the region generation portion generates the second diagnosis region by using a range not including the first diagnosis region, a range from which the second diagnosis region to be generated on the ultrasound image does not protrude, and a range in which the second diagnosis region is not provided on the side edges or the peripheral tissues of the first diagnosis region.

According to this configuration, possibility distribution can be generated by the range not including the first diagnosis region, the range from which the second diagnosis region to be generated on the ultrasound image does not protrude, and the range in which the second diagnosis region is not provided on the edges or the peripheral tissues of the first diagnosis region, and the second diagnosis region can be generated by using such possibility distribution.

In the ultrasound diagnostic apparatus of the present invention, the region generation portion generates the second diagnosis region by further using a range in which the second diagnosis region is not provided at a position with a depth larger than that of the first reference position.

According to this configuration, possibility distribution can be generated by adding the range in which the second diagnosis region is not provided at a position with a depth larger than that of the first reference position, and the second diagnosis region can be generated by using such possibility distribution.

In the ultrasound diagnostic apparatus of the present invention, the region generation portion generates the second diagnosis region by further using a straight line passing through the first reference position.

According to this configuration, possibility distribution can be generated by adding the straight line passing through the first reference position, and the second diagnosis region can be generated by using such possibility distribution.

In the ultrasound diagnostic apparatus of the present invention, the image generation portion further includes the calculation portion for calculating a ratio between image data of the ultrasound image representing the first diagnosis region and image data of the ultrasound image representing the second diagnosis region.

According to this configuration, a ratio between measured values of the image data in the two diagnosis regions can be displayed. At that time, since the two diagnosis regions can be generated semi-automatically, accuracy of the ratio of the calculated measured values can be improved without variation in the measured values of the image data in these diagnosis regions. As a result, the ratio of the measured values with less variation can be displayed.

In the ultrasound diagnostic apparatus of the present invention, in the region generation portion, the value of the second reference position of the second diagnosis region is given to a plurality of the ultrasound images, and the second diagnosis region is generated by using the values on the same position of the plurality of ultrasound images.

According to this configuration, even if more conditions are used, possibility distribution can be generated easily by using such conditions, and generation accuracy of the second diagnosis region can be improved by using such possibility distribution.

In the ultrasound diagnostic apparatus of the present invention, the region generation portion generates the first diagnosis region in a circle having a first radius from the first reference position using the first reference position as the center of the first diagnosis region and generates the second diagnosis region in a circle having a second radius from the second reference position using the second reference position as the center of the second diagnosis region.

According to this configuration, a region at an equal distance from the first reference position can be generated as the first diagnosis region, and a region at an equal distance from the second reference position can be generated as the second diagnosis region. At that time, since it is only necessary to hold only the radius value as a parameter of the diagnosis region to be generated, configuration can be simplified.

In the ultrasound diagnostic apparatus of the present invention, the region generation portion holds a radius of the second diagnosis region as a specified value in advance and uses the held specified value as the second radius.

According to this configuration, by specifying accuracy of the radius value which is the parameter of the second diagnosis region on the basis of experiences, generation accuracy of the second diagnosis region can be improved.

In the ultrasound diagnostic apparatus of the present invention, the calculation portion calculates the ratio on the basis of a statistic value including at least one of an average value, a median value, a mode value, a maximum value, and a minimum value of the image data.

According to this configuration, by arbitrarily selecting the statistic value including at least one of an average value, a median value, a mode value, a maximum value, and a minimum value of the image data in accordance with an application, the ratio of the measured values based on such statistic value can be calculated.

In the ultrasound diagnostic apparatus of the present invention, the first reference position is set by the control panel at a disease site of the displayed ultrasound image, and the image generation portion generates the second diagnosis region in a reference part of the disease site.

According to this configuration, a region of interest (ROI) can be automatically generated in the disease site and the reference part of the disease site, respectively. For example, an ROI can be generated in a tumor site and a fat part, respectively, and elasticity ratios at these parts can be displayed. As a result, benignity or malignancy of a tumor, necessity of a surgery and the like can be accurately determined.

### Embodiment 3

In an ultrasound diagnostic apparatus of Embodiment 3 of the present invention, the ROI generating methods of the above-described Embodiment 1 and Embodiment 2 are combined and moreover, it is configured such that appropriateness of the generated ROI can be evaluated, and necessary medication can be made easily. The ultrasound diagnostic apparatus according to Embodiment 3 includes an ultrasonic probe (hereinafter referred to as a probe) 51, a transmission/reception portion 52 for transmitting/receiving an ultrasound beam to/from an object, not shown, through the probe 51, a contrast image generation unit 53 for generating a contrast image on the basis of a reception beam signal subjected to reception processing in the transmission/reception portion 52, an elasticity image generation unit 54 for generating an elasticity image by acquiring an elasticity value of a tissue of the object on the basis of the reception beam signal, a display image generation unit 55 for synthesizing the contrast image and the elasticity image, an image display unit 56 for displaying an image synthesized in the display image generation unit 55, a control panel 57 having an input device such as a pointing device, and a control unit 58. The contrast image generation unit 53, the elasticity image generation unit 54, the display image generation unit 55, the image display unit 56, the control panel 57, and the control unit 58 are connected to a system bus 59 and are formed capable of transmission/reception of data such as an instruction signal, various types of data, and control data mutually through the system bus 59.

A feature of Embodiment 3 is a configuration of a region of interest generation unit 60. The region of interest generation unit 60 includes a reference ROI generation portion 61 connected to the system bus 59, a first ROI generation portion 62, a second ROI generation portion 63, an elasticity value calculation portion 64, an ROI evaluation portion 65, and an ROI modification portion 66. Each of these portions transmits/receives data such as an instruction signal, various types of data, and control data mutually through the system bus 59 and is formed capable of transmission/reception of the data between the contrast image generation unit 53, the elasticity image generation unit 54, and the control panel 57. Moreover, each of the portions constituting the region of interest generation unit 60 is configured to execute each function by a computer program. Moreover, the control unit 58 is configured to control each portion of the entire ultrasound apparatus and to execute control by the computer program. Moreover, the control unit 58 is constituted by using a calculation control device such as a CPU and the like, for example, and is configured to control synchronization of a series of processing from the control panel 57, the reference ROI generation portion 61, the first ROI generation portion 62, the second ROI generation portion 63, the elasticity value calculation portion 64, the ROI evaluation portion 65, the ROI modification portion 66, the display image generation unit 55, and the image display unit 56, when the measurement item or the ROI is set or changed.

The probe 51 converts a transmission signal given by the transmission/reception portion 52 to an acoustic signal and sends it to a diagnosis part of an object and converts the acoustic signal reflected by a biological tissue of the diagnosis part to an electric echo signal and transmits it to the transmission/reception portion 52. The probe 51 has a linear type, a convex type, a sector type and the like, and any one may be used. The transmission/reception portion 52 transmits/receives an ultrasound signal between the probe 51 and the diagnosis part of the diagnosing part by forming a transmission/reception beam and applies reception processing to the received reflected echo signal, generates a reception beam signal and gives it to the contrast image generation unit 53. The contrast image generation unit 53 forms a contrast image called a B image in general by those skilled in the art from the given reception beam signal and gives it to the display image generation unit 55. Moreover, the elasticity image generation unit 54 calculates an elasticity value (strain and elastic modulus) of the biological tissue corresponding to each of measurement points on the contrast image from the reception beam signal and generates elasticity frame data of the elasticity image on the basis of the elasticity value. The display image generation unit 55 synthesizes the elasticity image and the contrast image or forms the respective own display images and gives it to the image display unit 56 for display. Moreover, the display image generation unit 55 generates a profile figure expressing an ROI generated by the reference ROI generation portion 61, the first ROI generation portion 62, and the second ROI generation portion 63, superimposes it on the display image such as the synthesized image of the elasticity image and the contrast image and gives it to the image display unit 56 for display. The image display unit 56 is a display of an ultrasound diagnostic apparatus. The control panel 57 is a user interface for performing various operations of the ultrasound diagnostic apparatus. Particularly, the control panel 57 of Embodiment 3 includes a pointing device used for designating a position of the biological tissue on an image such as the contrast image displayed on the display of the ultrasound diagnostic apparatus. That is, the control panel 57 is formed by having an input device such as a keyboard, a trackball, a switch, a dial, a mouse, a touch panel and the like, for example. Moreover, the control panel 57 may be combined with sound input.

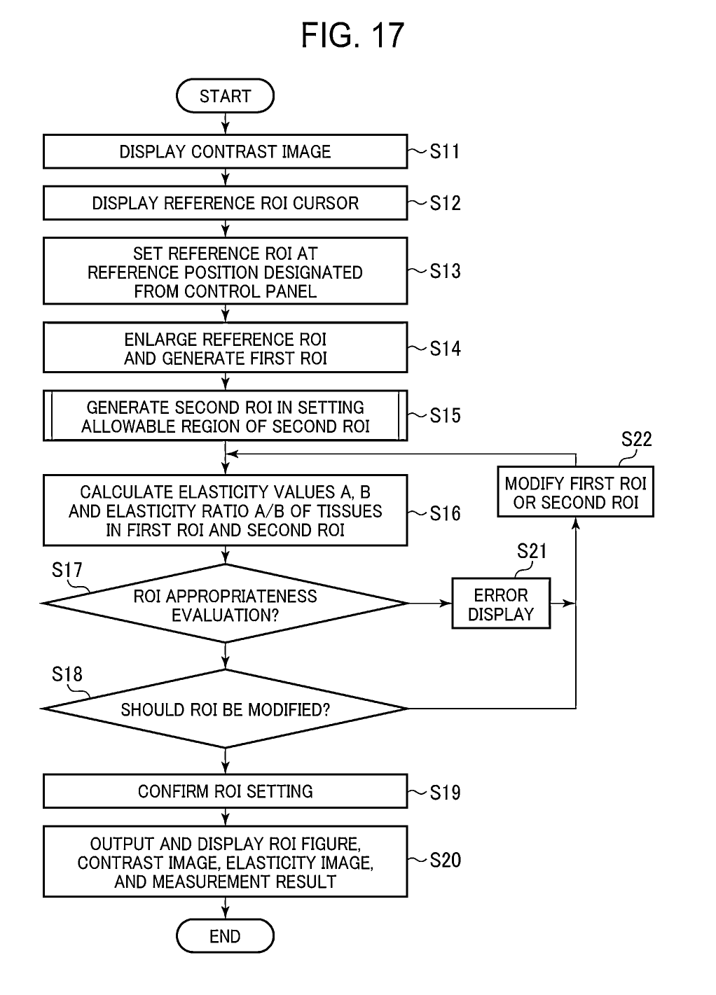

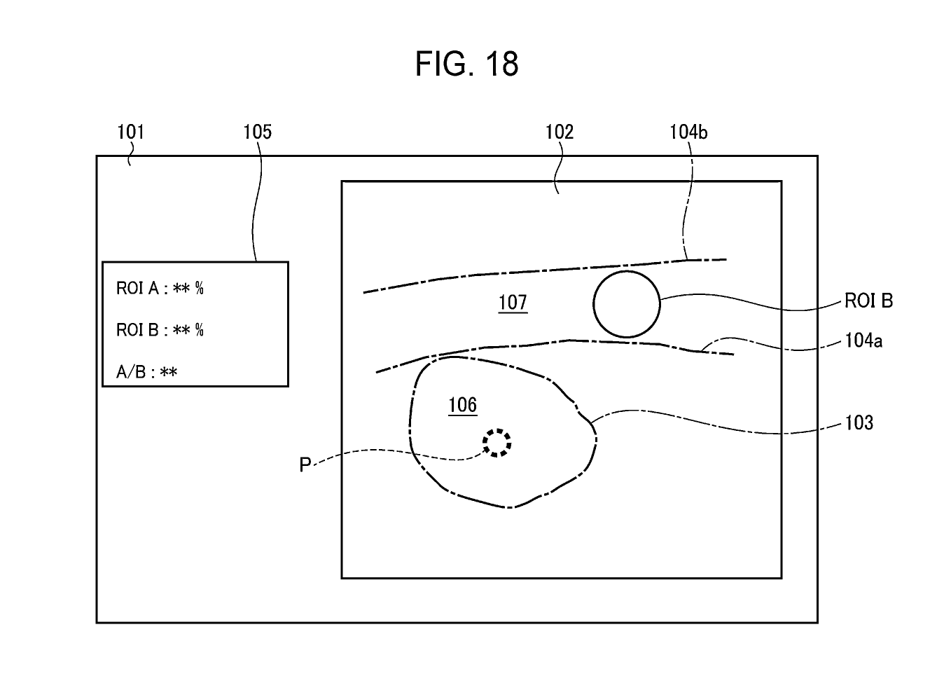

A configuration of each portion of the region of interest generation unit 60 of Embodiment 3 will be explained with a processing operation by referring to FIGS. 17 to 22. Each portion of the region of interest generation unit 60 is configured to generate and to set a region of interest in accordance with processing in a flowchart illustrated in FIG. 17 in collaboration with the control unit 58. On a display screen 101 of the image display unit 56, as illustrated in FIG. 18 as an example, a contrast image 102 is displayed. In this figure, one-dot chain lines 103, 104a, and 104b indicate a boundary from an adjacent biological tissue or a profile, respectively. Moreover, on the display screen 101, a calculation result 105 of the elasticity value and the elasticity ratio is displayed as measured values indicating a measurement result relating to measurement items. In Embodiment 3, a diagnosis target is a mammary tissue, for example, and is a tumor in which a first region 106 surrounded by the one-dot chain line 103 of the contrast image 102 is rendered in the tissue. Moreover, a second region 107 sandwiched by the one-dot chain lines 104a and 104b is a fat layer rendered in the tissue. Then, a case in which the elasticity values of the tumor and the fat and the elasticity ratios which are ratios of each of them are measured will be explained as an example. In the following, a region of interest and image information of measured values and the like generated at each portion are configured to be superimposed on the contrast image 102 in the display image generation unit 55 and displayed on the image display unit 56 and thus, explanation will be omitted as appropriate in explanation of each portion for simplification of the explanation.

### (Step S11)

As illustrated in the flowchart in FIG. 17, the control unit 58 starts region of interest setting processing on the basis of a region of interest setting start instruction inputted from the control panel 57. Then, as illustrated in FIG. 18, the contrast image 102 generated by the contrast image generation unit 53 is displayed by the image display unit 56 and frozen. At this time, the display image in which the contrast image and the elasticity image are superimposed can be freeze-displayed on the image display unit 56.

### (Step S12)

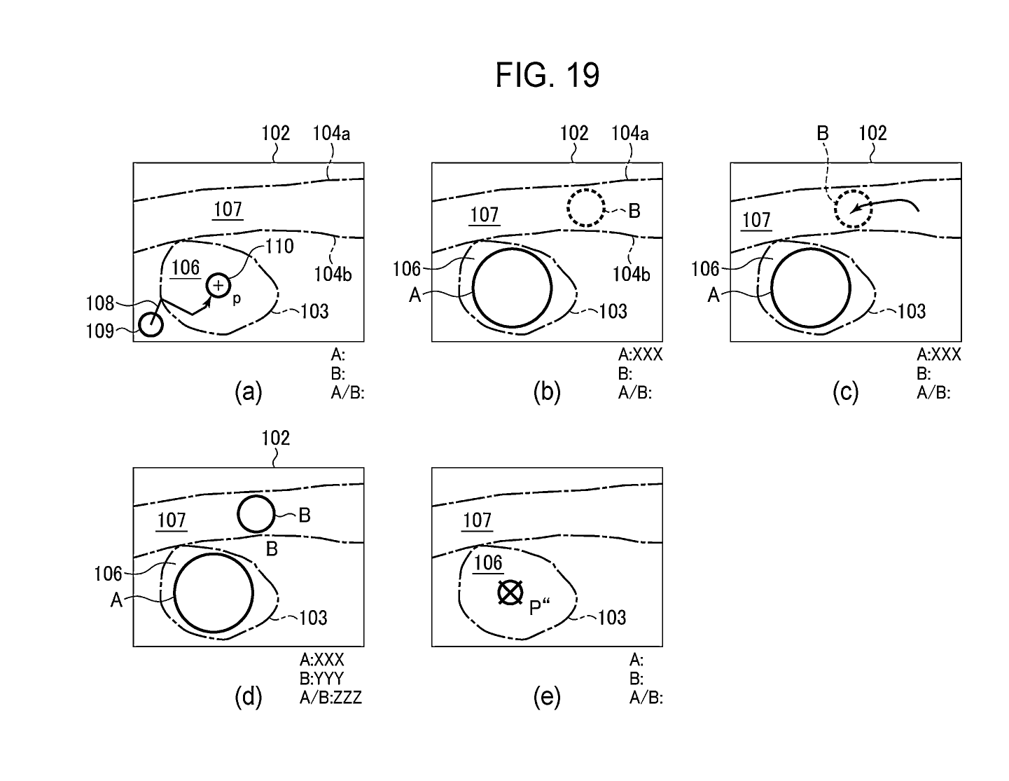

The reference ROI generation portion 61 generates a circular cursor with a minimum radius which becomes an initial ROI of the first ROI to be generated by the first ROI generation portion 62 as a reference ROIP and outputs figure data of the reference ROIP together with a display unit to the display image generation unit 55. Here, the minimum radius is set on the basis of a smallest number of the pixels included in the reference ROIP determined in advance in order to ensure calculation accuracy of the elasticity value. In the case of the reference ROI other than a circle, it is only necessary that an allowable minimum area is determined with the same concept and its shape is specified. As a result, as illustrated in FIG. 19(a), the reference ROIP is displayed at a predetermined position (lower left on the screen, for example) 109 of the contrast image 102.

### (Step S13)

The reference ROI generation portion 61 moves the cursor as indicated by an arrow 108 in accordance with an instruction from the control panel 57, and the reference ROIP is positioned at the specified reference position 110 in the first region 106 on the contrast image 102 (FIG. 19(a)). The reference ROI generation portion 61 sets the reference ROIP at the desired reference position 110 instructed by the pointing device provided on the control panel 57 during a process of moving the reference ROIP. The reference ROIP, here, is set to an allowable minimum area (a circle with the radius r₀) determined in advance.

### (Step S14)

The first ROI generation portion 62 fixes center coordinates of the reference ROIP which is a coordinate position specified from the control panel 57 to the reference position 110, enlarges the radius r of the reference ROIP (area) and generates a first ROIA. This enlargement processing can be performed such that the examiner enlarges it to an arbitrary size by a cursor operation or the like from the control panel 57 while watching the display screen 101, but in Embodiment 3, the first ROI generation portion 62 performs enlargement automatically. Since the first ROI generation portion 62 is configured similarly to the ROI generation unit 12 in FIG. 2 of Embodiment 1, refer to Embodiment 1 for details. First, as illustrated in the flowchart in FIG. 3, filtering processing such as speckle removal processing is applied to the contrast image 102 (S4 in FIG. 3). Subsequently, on the basis of a change in a pixel value in the two-dimensional direction of the contrast image 102 from a center P0 of the reference ROIP, a tissue boundary 103 of the first region in which the reference ROIP is set is detected (S5 in FIG. 3). Then, by setting a plurality of center candidate points pn in the reference ROIP ((see FIG. 5), a shortest distance from each center candidate point pi to the tissue boundary 103 (reference numeral 23 in FIG. 2) is acquired, respectively (S6 in FIG. 3). Moreover, a circle having a shortest distance which is the longest around the center candidate point pi whose shortest distance is the longest as a radius is acquired (S7, S8 in FIG. 3). Then, a circle or a polygonal region inscribed in the circle is generated as the first ROIA (S9 in FIG. 3). As described above, the first ROIA which is enlarged so as to abut against the tissue boundary 103 of the first region is generated by the first ROI generation portion 26 and displayed on the display screen 101 as illustrated in FIG. 19(b).

### (Step S15)

The second ROI generation portion 63 includes the second ROI parameter storage portion 332, the possibility distribution generation portion 333, and the second ROI generation portion 334 of Embodiment 2 illustrated in FIG. 11. That is, a second ROIB is automatically generated in the second region 107 of the biological tissue different from the biological tissue of the first region 106 on the contrast image 102. An area and a shape of the second ROIB are set in advance, and explanation will be made in this embodiment assuming that it is set in a circular region with a radius rb.

Regarding the second ROI generation portion 63, a generation allowed region satisfying conditions that the second ROIB is on the contrast image 102 and the range does not include the first ROIA, that it is the range in which the second ROIB does not protrude from the contrast image 102, and that the range does not include the edge of the first ROIA and the peripheral tissue of the first region 106 is set by the possibility distribution generation portion 333 in FIG. 11. Then, the second ROI generation portion 63 searches and determines a position where the second ROIB is to be generated in the generation allowed region. On the basis of the center of the second ROIB satisfying the above conditions, the generation allowed region is acquired on the contrast image 102 and stored in the memory of the second ROI generation portion 63, and the center of the second ROIB is set in the generation allowed region. Here, explanation is made assuming that the area and the shape of the second ROIB are set in advance, but the area set in advance may be applied on the basis of this center or may be automatically enlarged as in step S14 so as to set the ROI. If automatic enlargement of the second ROI is interlocked similarly to the first ROI, the first ROI and the second ROI have the same number of pixels at all times, which contributes to accurate calculation of the elasticity ratio.

### (Step S16)

The elasticity value calculation portion 64 calculates elasticity values A, B in the first ROIA and the second ROIB, respectively. That is, elasticity data of the elasticity image corresponding to the contrast image 102 displayed on the display screen 101 is extracted by accessing an elasticity frame data memory of the elasticity image generation unit 54. Then, the elasticity value is extracted by the unit of pixels, for example, and the elasticity values A, B totaling the elasticity value of the plurality of pixels present in the first ROIA and the second ROIB are calculated, respectively. Instead of this, an average value of the elasticity values of the plurality of pixels may be used. Moreover, an elasticity ratio A/B which is a ratio between the elasticity values A, B is calculated. It is an aim of the region of interest generation unit 60 to generate and set the first ROIA and the second ROIB in order to improve accuracy of a measurement result of this elasticity ratio A/B and to obtain the measurement result with high reproducibility.

### (Step S17)

The ROI evaluation portion 65 evaluates whether or not the first ROIA and the second ROIB are appropriate on the basis of the respective elasticity values A, B of the first ROIA and the second ROIB or the elasticity ratio A/B. That is, the ROI evaluation portion 65 determines whether or not the respective elasticity values A, B calculated in the elasticity value calculation portion 64 is within a set range determined in advance or whether or not their elasticity ratio A/B is within a set range determined in advance and evaluates whether generation of the first ROIA or the second ROIB is appropriate or not. Here, an idea on the set range for determining appropriateness of the elasticity values A, B will be explained. For example, since the first region is set to a region with a hard tissue such as a tumor, the elasticity value A becomes a small value. On the other hand, since the second region is set to a region with a soft tissue such as a fat, for example, the elasticity value B becomes a relatively large value. Then, if the elasticity value A is too small in view of an empirical value, it can be considered that an area of the generated first ROIA is too small and the number of samples is small. To the contrary, if the elasticity value A is too large, it can be considered that the area of the generated first ROIA is too large and it contains a soft region other than the hard region such as a tumor. On the other hand, the second region is a tissue having relatively uniform elasticity such as a fat layer, but it can be considered that a set position of the generated second ROIB is inappropriate and it contains a hard tissue and the like. Then, it is determined whether or not the respective elasticity values A, B are within the determined set range, respectively, so as to determine wither or not the generation of the first ROIA or the second ROIB is appropriate. Similarly, since the elasticity ratio A/B which is a final result is also subjected to an influence of the elasticity values A, B, it is determined whether or not they are within the set range determined in advance, and it is determined whether or not the generation of each of the ROIA, the ROIB is appropriate.

### (Steps S18 to S20)

If the determination at step S17 is appropriate, a message reading “Confirm ROI setting?”, for example, is displayed on the display screen 101 from the ROI evaluation portion 65 at step S17. In response to that, if ROI setting confirmation is inputted from the control panel 57, it is determined at step S18 that no modification will be made, and the routine proceeds to step S19. Then, at step S19, an ROI setting confirmation instruction is inputted to the control unit 58, the control unit 58 has the first ROIA, the second ROIB, the contrast image, the elasticity image, the elasticity values A, B and the elasticity ratio A/B which are the measurement results having been confirmed displayed on the display screen 101, and region of interest setting is finished.

### (Step S21)

If the appropriateness evaluation of the ROI at step S17 is not acceptable, error display is made (S21), and the routine proceeds to step S22. Moreover, if an instruction to modify the first ROIA and/or the second ROIB is inputted from the control panel 57 for the other reasons such as an intension of the examiner or the like at the determination at step S18, the routine also proceeds to step S22.

The ROI modification portion 66 modifies at least either one of the first ROIA and the second ROIB in collaboration processing with the control panel 57, returns to step S16, calculates the elasticity values A, B and the elasticity ratio A/B and repeats the appropriateness evaluation of the ROI, and if appropriateness evaluation can be obtained, as described above at step 10, the measurement result and the like are displayed, and the processing is finished.

### (Step S22)

Modification processing in the ROI modification portion 66 has four modes. That is, in order to solve the error, (1) modification of moving the position of the second ROIB; (2) modification of enlarging or contracting the second ROIB; (3) modification of moving the position of the first ROIA, and (4) modification of enlarging or contracting the first ROIA. The ROI modification portion 66 can be configured to automatically change to a modification mode of the first ROIA or the second ROIB which was not displayed depending on a cause of the error. Moreover, the examiner can also select the modification mode freely.

The modification mode (1) will be explained by referring to FIG. 19. For example, as illustrated in FIG. 19(b), if the elasticity value A could be measured but the elasticity value B could not be measured, a value is not displayed for the elasticity value B on the display screen 102. Moreover, the second ROIB immediately after being set at step S15 is indicated by a dotted line. In this case, it is an error, and processing at step S22 is started via step S21. First, in the display state in FIG. 19(a), by clicking the reference ROIP which is a cursor in the vicinity of the center of the first region 106, the first ROIA is indicated by a solid line and the second ROIB by a dotted line as illustrated in FIG. 19(b), and thus, it is known that modification of the second ROIB is needed. Thus, the ROI modification portion 66 reads the coordinates of the second ROIB, assigns a cursor operation function to the control panel 57, and as illustrated in FIG. 19(c), the second ROIB is made movable by the cursor operation. Then, when the movement of the second ROIB by the operation of the control panel 57 is finished, the control unit 58 causes the elasticity value calculation portion 64 to execute processing (step S16 in FIG. 17) and to calculate the elasticity values A, B and the elasticity ratio A/B. Subsequently, the ROI evaluation portion 65 is made to execute processing (S17 in FIG. 17). As a result, if the ROI evaluation becomes appropriate, as illustrated in FIG. 19(d), the second ROIB after the examiner moved is indicated by a solid line circle, and values are displayed for the elasticity values A, B and the elasticity ratio A/B. If the ROI evaluation is not appropriate even after the modification, as illustrated in FIG. 19(e), a cross mark is displayed indicating error display on the reference ROIP, for example.

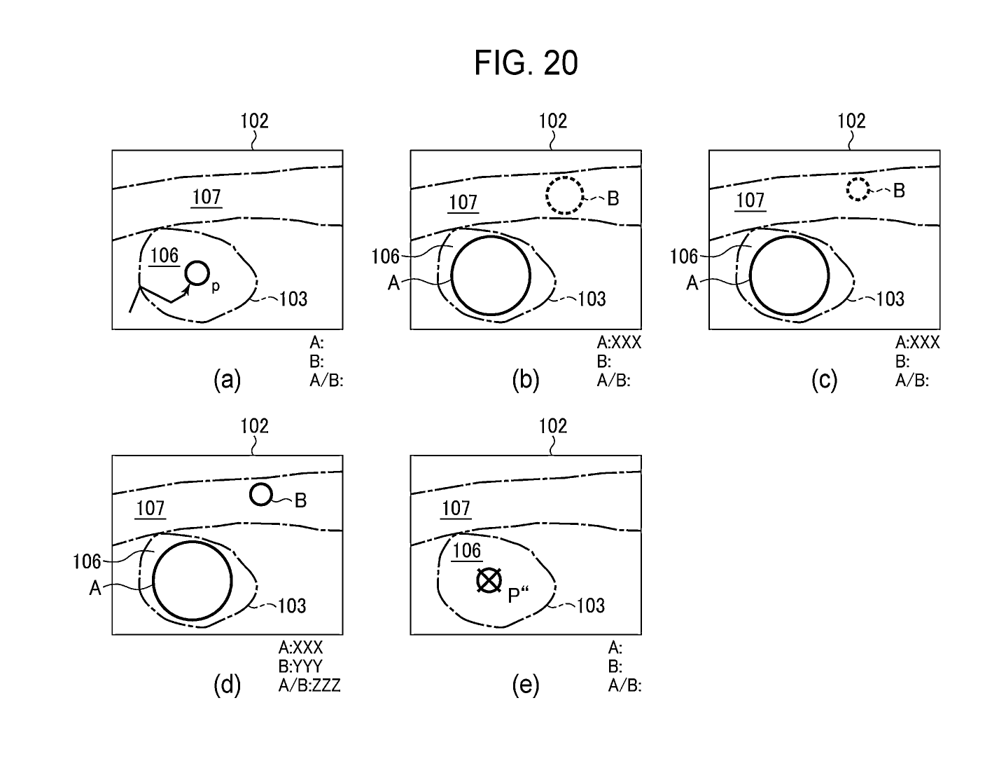

The modification mode (2) will be explained by referring to FIG. 20. In the display state in FIG. 20(a), by clicking the reference ROIP which is a cursor in the vicinity of the center of the first region 106, the display state changes to that in FIG. 20(b), the first ROIA is indicated by a solid line and the second ROIB by a dotted line and it is known that modification of the second ROIB is needed. Thus, by contracting the diameter of the second ROIB as in FIG. 20(c), a circle of the second ROIB is indicated by a solid line as illustrated in FIG. 20(d) and values are also displayed for the elasticity values A, B and the elasticity ratio A/B. As a result, appropriate generation and setting of the ROI are finished. If the ROI evaluation is not appropriate even after the modification, as illustrated in FIG. 20(e), a cross mark is displayed indicating error display on the reference ROIP, for example.

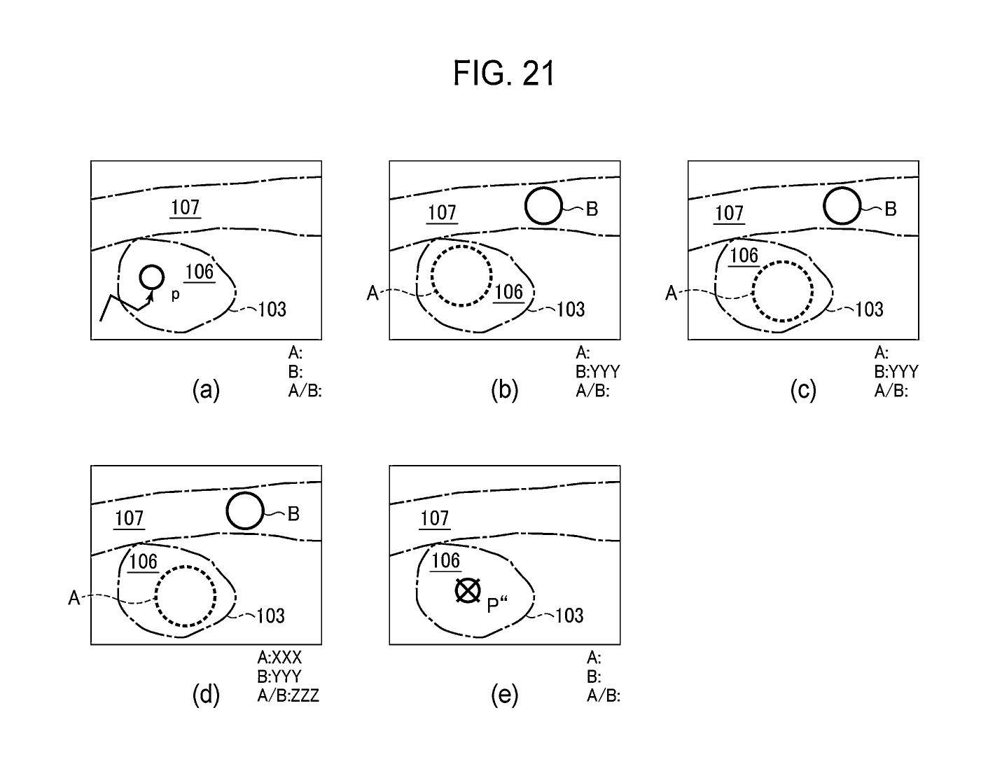

The modification mode (3) will be explained by referring to FIG. 21. In the display state in FIG. 20(a), by clicking the reference ROIP which is a cursor in the vicinity of the center of the first region 106, the display state changes to that in FIG. 21(b), the first ROIA is indicated by a dotted line and the second ROIB by a solid line and it is known that modification of the first ROIA is needed. Thus, by moving the first ROIA as in FIG. 21(c), a circle of the first ROIA is indicated by a solid line as illustrated in FIG. 21(d) and values are also displayed for the elasticity values A, B and the elasticity ratio A/B. As a result, appropriate generation and setting of the ROI are finished. If the ROI evaluation is not appropriate even after the modification, as illustrated in FIG. 21(e), a cross mark is displayed indicating error display on the reference ROIP, for example.

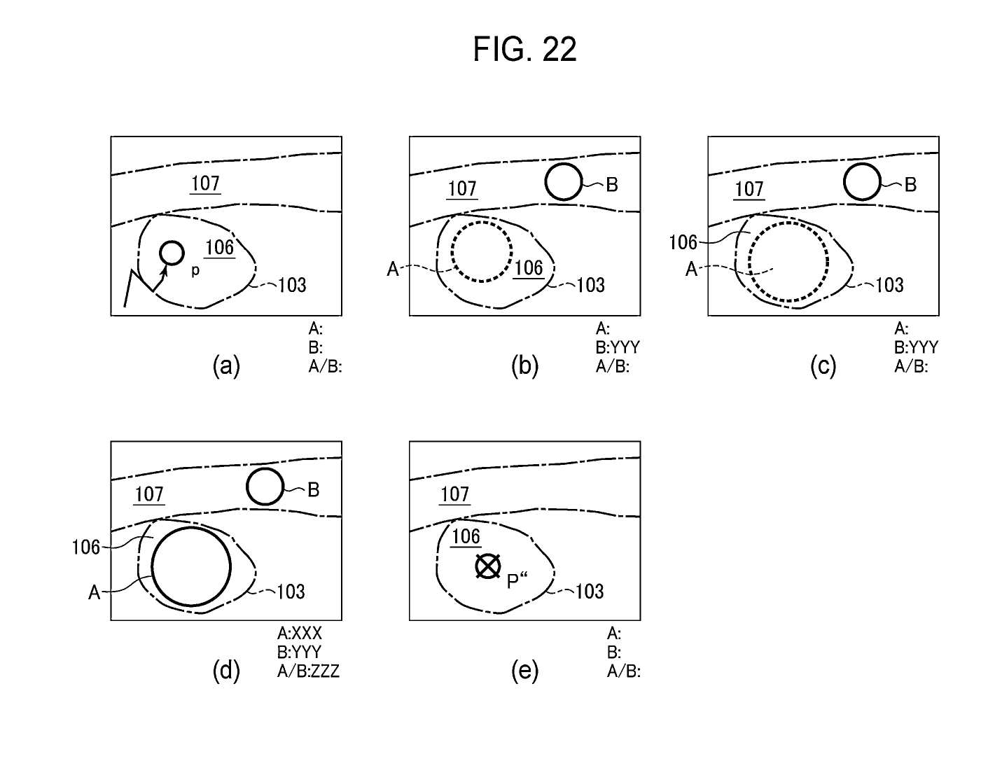

The modification mode (4) will be explained by referring to FIG. 22. In the display state in FIG. 22(a), by clicking the reference ROIP which is a cursor in the vicinity of the center of the first region 106, the display state changes to that in FIG. 22(b), the first ROIA is indicated by a dotted line and the second ROIB by a solid line and it is known that modification of the first ROIA is needed. Thus, by enlarging the first ROIA as in FIG. 22(c), a circle of the first ROIA is indicated by a solid line as illustrated in FIG. 22(d) and values are also displayed for the elasticity values A, B and the elasticity ratio A/B. As a result, appropriate generation and setting of the ROI are finished. If the ROI evaluation is not appropriate even after the modification, as illustrated in FIG. 22(e), a cross mark is displayed indicating error display on the reference ROIP, for example.

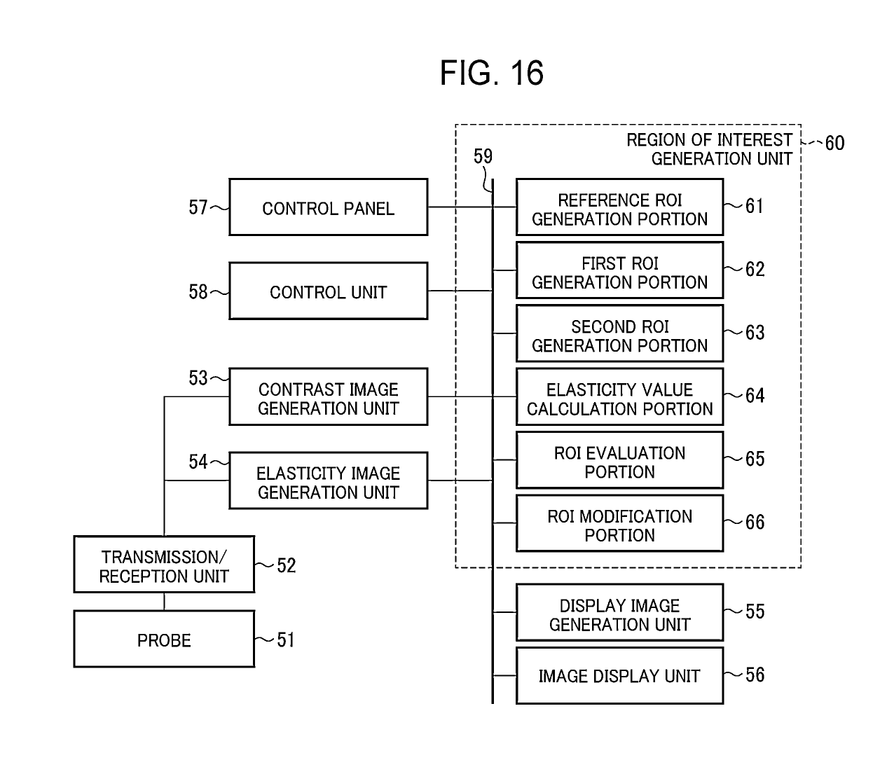

As described above, according to Embodiment 3, the examiner can generate and set the position and the size (area) of the plurality of ROIs with fewer procedures and less time, and thereby an elasticity value with high accuracy and reproducibility can be measured. Moreover, the modification of the ROI can be started without extra operation by the examiner. With the region of interest generation unit 60 in FIG. 16 of this Embodiment 3, effects such as high operability, reduced labor of the examiner, and improvement of inspection efficiency can be obtained.

Regarding the reference ROI generation portion 61 of this Embodiment 3, the example in which the reference ROI with an allowable minimum area (a circle with the radius r₀) determined in advance at step S13 in FIG. 17 is set at a designated position is explained. Instead of this, the reference ROI can also be generated automatically. That is, the reference ROI generation portion 61 can read the number of minimum pixels of the reference ROI set in advance from the memory and generate the circle with the radius r₀ of the reference ROI on the basis of the image data of the contrast image 102 at the position designated by the cursor.

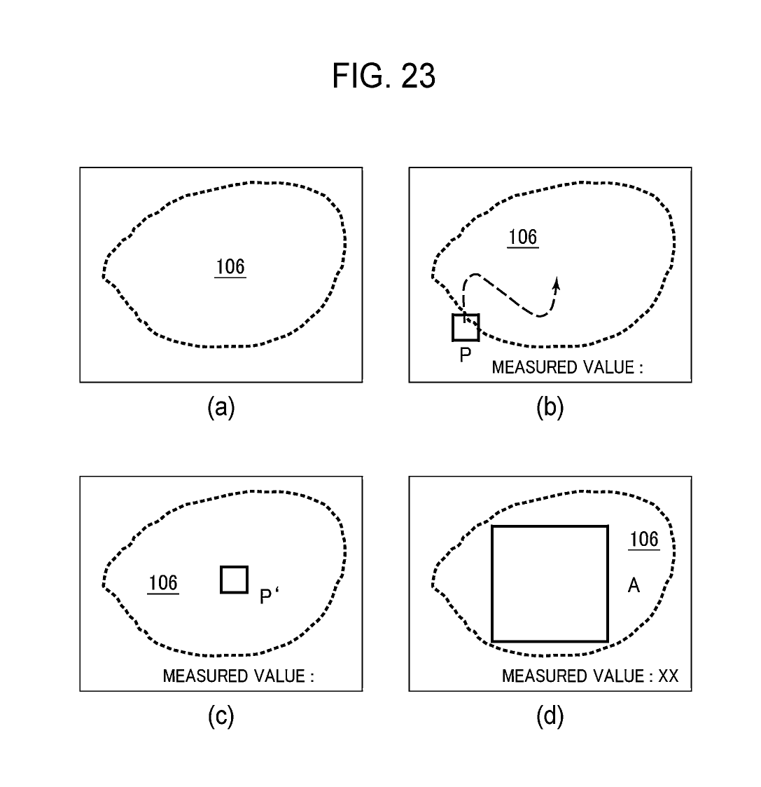

Moreover, in Embodiment 3, the example in which the shapes of the first ROI and the second ROI are circular is explained, but as illustrated in FIG. 23, a rectangular ROI can be used. Moreover, the shape of the ROI in the present invention is not limited to circular or rectangular, and an arbitrary closed two-dimensional figure such as an oval and a polygon can be applied. In short, it is only necessary that a shape in which the number of pixels capable of sampling of an elasticity value can be made as large as possible, conforming to the tissue structure to be measured. Since the size indicating the pixel is different depending on a display depth in an ultrasound image, when the ROI dimension of the allowable minimum area is to be determined, the size may be determined by the unit of m by a m/pixel value of the ultrasound image.

Moreover, in Embodiment 3, explanation was made such that, in the enlargement processing of the first ROIA at step S14, the plurality of center candidate points Pn are set in the reference ROIP in accordance with Embodiment 1. This center candidate point Pn does not have to have coordinates in the vicinity of the center of the reference ROIP but can be set arbitrarily. Moreover, a set position of the center candidate point Pn can be made into a figure, superimposed on the reference ROIP and superimposed and displayed on the contrast image and the elasticity image. As a result, the examiner can confirm on the basis of what plurality of center candidate points Pn that the first ROIA was enlarged. Moreover, the center candidate point Pn can be set in the region touched by the examiner using a touch panel or the like constituting the control panel 57, for example, instead of automatic determination by the first ROI generation portion 62.

Moreover, by juxtaposing and displaying the contrast image and the elasticity image with the same cross section on the display screen 101 of the image display unit 56, the designated position of the reference ROI or the reference ROI can be displayed in different display modes (different shapes, for example) at the same time.

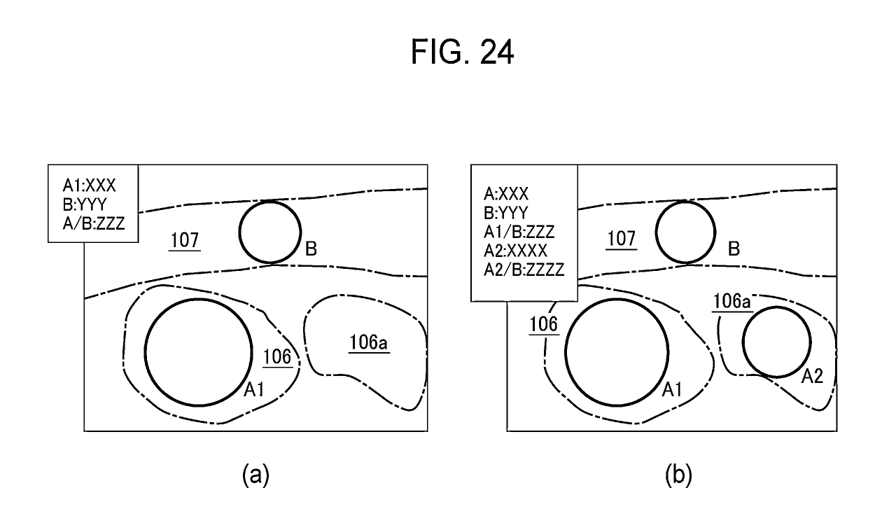

Here, in Embodiment 3, the first ROIA can be generated in plural and set. This will be explained by referring to FIG. 24. That is, in the above-described example, explanation was made such that one first ROIA is generated by the first ROI generation portion 62, but two or more first ROIs can be generated by the first ROI generation portion 62. In this case, the first ROI generation portion 62 repeats steps S12 to S14 in FIG. 17 and generates a first ROIA1 in the first region 106 as illustrated in FIG. 24(a) and generates a first ROIA2 in a third region 106a. The generation procedure of each ROI is similar to the above-described examples. When three or more first ROIA1 to A3 are to be generated, they can be generated similarly by repeating steps S12 to S14.

When a plurality of the first ROIA are generated, the elasticity value calculation portion 64, the ROI evaluation portion 65, and the ROI modification portion 66 calculate elasticity values A1, A2 and elasticity ratios A1/B, A2/B for the two first ROIA1 and first ROIA2, respectively, and display them as a measurement result on the display screen 101. Moreover, the ROI evaluation portion 65 makes evaluation for the first ROIA1 and the first ROIA2, respectively. Moreover, the ROI modification portion 66 can apply modification processing corresponding to the above-described modification processing mode to the first ROIA1 or the first ROIA2 subjected to an error in accordance with the appropriateness evaluation of the first ROIA1 and the first ROIA2.

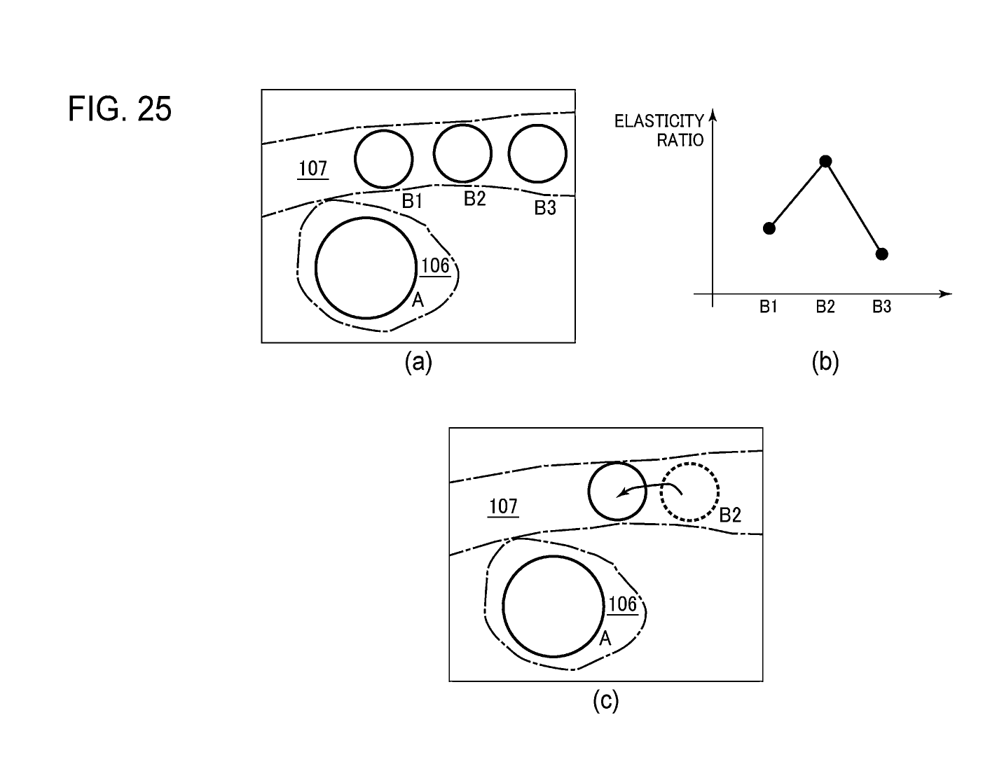

Moreover, an example in which a plurality of the second ROIB are generated and set will be explained by referring to FIG. 25. In the above-described example, the case in which the one second ROI is generated by the second ROI generation portion 63 was explained, but the second ROI generation portion 63 can generate a plurality of the second ROIBs. In this case, the second ROI generation portion 63 can repeat step S15 in FIG. 17 and set the plurality of (three in the illustrated example) second ROIB1 to B3 in the same second region 107, as illustrated in FIG. 25(a). Since the second ROIB is configured to be automatically generated and set, by inputting the number of setting of the second ROIB from the control panel 57 as the generation condition, the second ROI generation portion 63 makes determination as appropriate and determines positions so that the second ROIB1 to B3 are not overlapped and arranges them.

For the second ROIB1 to B3 set as above, the elasticity value calculation portion 64 calculates elasticity values B1, B2, B3 and elasticity ratios A/B1, A/B2, A/B3, respectively, and displays them on the display screen 101 as a measurement result. Moreover, the elasticity value calculation portion 64 creates a graph of the elasticity ratio associated with the second ROIB1 to B3 as illustrated in FIG. 25(b) so that the elasticity ratios A/B1, A/B2, A/B3 can be compared and displays it on the display screen. As a result, the examiner can determine appropriateness of the elasticity ratios A/B1, A/B2, A/B3.

Moreover, the ROI evaluation portion 65 makes evaluation for the second ROIB1 to B3, respectively. If the evaluation is an error, the ROI modification portion 66 changes to the above-described modification processing mode in accordance with the evaluation results of the first ROIA and the second ROIB1 to B3. In response to that, the image on which the second ROIB1 to B3 subjected to an error are displayed is displayed, and thus, the modifications processing can be executed by moving the position of the second ROIB2, for example, as illustrated in FIG. 25(c). In the case of this modification, the second ROIB1 to B3 to be modified can be made selectable. This selection can be made by the cursor from the control panel 57 but may be directly selected from the above-described touch panel or can be also selected by a toggle method.

According to this Embodiment 3, only by setting by the examiner the center position of the first ROIA as the reference position (first reference position), the first ROIA and the second ROIB can be both generated automatically. In short, the two ROIs used for the elasticity ratio calculation can be semi-automatically generated. Therefore, the elasticity values acquired in the first ROIA and the second ROIB are not varied and as a result, accuracy of the calculated elasticity ratio can be improved. As a result, benignity or malignancy of a tumor, necessity of a surgery and the like, for example, can be accurately determined.

As described above, the method for setting regions of interest of the present invention according to Embodiment 3 is a method for setting regions of interest for setting the first region of interest in the first region and the second region of interest in the second region in order to calculate a ratio of elasticity values of the first region of an ultrasound image obtained by an ultrasound diagnostic apparatus and the second region with the biological tissue different from that of the first region, wherein a reference region of interest with an area determined in advance is generated and set at a position designated as the first region on the ultrasound image, the first region of interest is generated and set by enlarging the reference region of interest, the second region of interest is generated and set in the second region, elasticity values of the first region of interest and the second region of interest set, respectively, are calculated, respectively, evaluation is made on whether or not generation of the first region of interest and the second region of interest is appropriate on the basis of the respective elasticity values or their ratio, and at least one of the first region of interest and the second region of interest is modified in accordance with the evaluation.

Moreover, the ultrasound diagnostic apparatus performing the method for setting regions of interest of the present invention according to Embodiment 3 includes a transmission/reception portion for transmitting/receiving an ultrasound beam between an object and itself through an ultrasonic probe, a contrast image generation portion for generating a contrast image on the basis of a reception beam signal subjected to reception processing in the transmission/reception portion, an elasticity image generation portion for generating an elasticity image by acquiring an elasticity value of a tissue of the object on the basis of the reception beam signal, a region of interest generation portion for setting a region of interest in the contrast image, a display image generation portion for synthesizing the contrast image, the elasticity image, and the figure of the region of interest, an image display unit for displaying an image synthesized in the display image generation portion, and a control panel having a pointing device, wherein the region of interest generation portion includes a reference region of interest generation portion for setting a reference region of interest with an area determined in advance in a first region on the contrast image designated by the pointing device, a first region of interest generation portion for generating a first region of interest by enlarging the reference region of interest, a second region of interest generation portion for generating a second region of interest in the second region with a biological tissue different from the biological tissue of the first region on the contrast image, an elasticity value calculation portion for calculating elasticity values of the first region of interest and the second region of interest, respectively, and an evaluation portion for evaluating whether or not the first region of interest and the second region of interest are appropriate on the basis of the respective elasticity values of the first region of interest and the second region of interest or their ratios, and the first region of interest generation portion and the second region of interest generation portion include a region of interest modification portion for modifying at least one of the first region of interest and the second region of interest in accordance with evaluation of the evaluation portion.

In this case, the region of interest modification portion can modify a position or an area of at least one of the first region of interest and the second region of interest. Moreover, the evaluation portion can evaluate whether or not generation of the first region of interest and the second region of interest is appropriate by whether or not the respective elasticity values of the first region of interest and the second region of interest calculated in the elasticity value calculation portion are within the set range or by whether or not the ratios of their elasticity values are within the set range.

Moreover, the second regions of interest are generated in plural and set, and the elasticity value calculation portion calculates a ratio of the elasticity values corresponding to the plurality of the second regions of interest, generates a graph and displays it on the image display unit so that one of the second regions of interest can be formed to be selectable by the pointing device.

Moreover, the first regions of interest are generated in plural and set, and the elasticity value calculation portion can calculate a ratio of the elasticity values corresponding to the plurality of the first regions of interest and can display it on the image display unit so that comparison can be made. Furthermore, when the evaluation portion evaluates that generation of the first region of interest and the second region of interest is not appropriate, that fact (error display by a message or a cross mark, for example) can be displayed on the image display unit.

The first region of interest generation portion of this Embodiment 3 includes a tissue boundary detection portion for detecting a tissue boundary of the first region on the basis of a change in a pixel value in a two-dimensional direction of the contrast image from a set position of the reference region of interest, a minimum distance calculation portion for setting a plurality of center candidate points in the reference region of interest and acquiring a shortest distance from each of the center candidate points to the tissue boundary, respectively, and a maximum distance calculation portion for acquiring a circle having the shortest distance which is the longest thereof around the center candidate point with the shortest distance which is the longest as a radius, and the circle or a polygonal region inscribed in the circle can be set as the first region of interest.

The ultrasound diagnostic apparatus according to claim 2, wherein the second region of interest generation portion generates the second region of interest in a range not including the first region of interest, a range in which the second region of interest does not protrude from the contrast image, and a range not including an edge of the first region of interest and a peripheral tissue of the first region on the contrast image.

Moreover, the present invention is not limited to the above-described embodiments and is capable of change/variation within a range described in claims.

As described above, according to the present invention, since a region of interest (ROI) can be automatically set, an elasticity ratio of a biological tissue can be acquired uniformly, and reliability of clinical data relating to elasticity can be improved. The clinical data with high reliability enables accurate diagnosis in individual diagnoses and narrowing of standard values relating to the diagnosis. Moreover, the present invention is not limited to the above-described embodiments and it is obvious that those skilled in the art can conceive of various change examples or modification examples within a range of a technical idea disclosed herein, and it is understood that they naturally belong to the technical scope of the present invention.

### REFERENCE SIGNS LIST

2, 21, 51 Ultrasonic probe
3 Transmission unit
4 Reception unit
5 Phasing addition circuit
6, 31, 53 Contrast image generation unit
7, 35, 55 Display image generation unit
8, 56 Image display unit
9 Elasticity calculation unit
10, 32, 54 Elasticity image generation unit
11 Apparatus control/interface unit
12 ROI generation unit
13 ROI image generation unit

1. A method for setting regions of interest for setting a first region of interest in a first region and a second region of interest in a second region in order to calculate a ratio of elasticity values of the first region of an ultrasound image obtained by an ultrasound diagnostic apparatus and the second region with a biological tissue different from that of the first region, wherein
a reference region of interest with an area determined in advance is generated and set at a position designated as the first region on the ultrasound image;
the first region of interest is generated and set by enlarging the reference region of interest;
the second region of interest is generated and set in the second region;
elasticity values of the first region of interest and the second region of interest set, respectively, are calculated, respectively;
evaluation is made on whether or not generation of the first region of interest and the second region of interest is appropriate on the basis of the respective elasticity values or their ratio; and
at least one of the first region of interest and the second region of interest is modified in accordance with the evaluation.

2. An ultrasound diagnostic apparatus, comprising:
a transmission and reception unit configured to transmit and receive an ultrasound beam between an object and itself through an ultrasonic probe;
a contrast image generation unit configured to generate a contrast image on the basis of a reception beam signal subjected to reception processing in the transmission and reception unit;
an elasticity image generation unit configured to generate an elasticity image by acquiring an elasticity value of a tissue of the object on the basis of the reception beam signal;
a region of interest generation unit configured to set a region of interest in the contrast image;
a display image generation unit configured to synthesize the contrast image, the elasticity image, and the figure of the region of interest;
an image display unit configured to display an image synthesized in the display image generation unit; and
a control panel having a pointing device, wherein
the region of interest generation unit includes:
a reference region of interest generation portion for setting a reference region of interest with an area determined in advance in a first region on the contrast image designated by the pointing device;
a first region of interest generation portion for generating a first region of interest by enlarging the reference region of interest;
a second region of interest generation portion for generating a second region of interest in the second region with a biological tissue different from the biological tissue of the first region on the contrast image;
an elasticity value calculation portion for calculating elasticity values of the first region of interest and the second region of interest, respectively; and
an evaluation portion for evaluating whether or not the first region of interest and the second region of interest are appropriate on the basis of the respective elasticity values of the first region of interest and the second region of interest or their ratios; and
the first region of interest generation portion and the second region of interest generation portion include a region of interest modification portion for modifying at least one of the first region of interest and the second region of interest in accordance with evaluation of the evaluation portion.

3. The ultrasound diagnostic apparatus according to claim 2, wherein
the region of interest modification portion modifies a position or an area of at least one of the first region of interest and the second region of interest.

4. The ultrasound diagnostic apparatus according to claim 2, wherein
the evaluation portion evaluates whether or not generation of the first region of interest and the second region of interest is appropriate by whether or not the respective elasticity values of the first region of interest and the second region of interest calculated in the elasticity value calculation portion are within set ranges or by whether or not the ratio of their elasticity values is within a set range.

5. The ultrasound diagnostic apparatus according to claim 2, wherein
the second regions of interest are generated in plural and set; and
the elasticity value calculation portion calculates a ratio of the elasticity values corresponding to the plurality of the second regions of interest, generates a graph and displays it on the image display unit, so that one of the second regions of interest is formed to be selectable by the pointing device.

6. The ultrasound diagnostic apparatus according to claim 2, wherein
the first regions of interest are generated in plural and set; and
the elasticity value calculation portion calculates a ratio of the elasticity values corresponding to the plurality of the first regions of interest and displays it on the image display unit so that comparison can be made.

7. The ultrasound diagnostic apparatus according to claim 2, wherein
when the evaluation portion evaluates that generation of the first region of interest and the second region of interest is not appropriate, that fact (a message thereof (cross mark, for example)) is displayed on the image display unit.

8. The ultrasound diagnostic apparatus according to claim 2, wherein
the first region of interest generation portion includes:
a tissue boundary detection portion for detecting a tissue boundary of the first region on the basis of a change in a pixel value in a two-dimensional direction of the contrast image from a set position of the reference region of interest;
a minimum distance calculation portion for setting a plurality of center candidate points in the reference region of interest and acquiring a shortest distance from each of the center candidate points to the tissue boundary, respectively; and
a maximum distance calculation portion for acquiring a circle having the shortest distance which is a longest thereof around the center candidate point with the shortest distance which is the longest as a radius; and
the circle or a polygonal region inscribed in the circle can be set as the first region of interest.

9. The ultrasound diagnostic apparatus according to claim 2, wherein
the second region of interest generation portion generates the second region of interest in a range not including the first region of interest, a range in which the second region of interest does not protrude from the contrast image, and a range not including an edge of the first region of interest and a peripheral tissue of the first region on the contrast image.

10. The ultrasound diagnostic apparatus according to claim 6, wherein
in the second region of interest, a shape and an area are set in advance.

11. An ultrasound diagnostic apparatus comprising:
an ultrasound image generation unit configured to generate an ultrasound image on the basis of a received reflected echo signal by transmitting and receiving ultrasonic waves to and from an object;
an image display unit configured to display the ultrasound image;
an input device for setting a designated region by a point or a region in a notable tissue of the ultrasound image displayed on the image display unit;
a tissue boundary detection unit configured to detect a tissue boundary on the basis of a change in a pixel value in a two-dimensional direction of the ultrasound image;
a minimum distance calculation unit configured to acquire a shortest distance from each of the candidate points to the tissue boundary, respectively;
a maximum distance calculation unit configured to acquire a circle having a shortest distance which is a longest around the candidate point with the shortest distance which is the longest thereof as a radius; and
a region of interest setting unit configured to set a circle or a polygonal region inscribed in the circle as the region of interest.

12. An ultrasound diagnostic apparatus comprising:
a probe configured to transmit an ultrasonic wave to an object and to receive a reflected signal from the object;
a transmission and reception unit configured to transmit and receive the ultrasonic wave by driving the probe and for executing signal processing of the reflected signal;
an image generation unit configured to generate an ultrasound image by using the reflected signal subjected to the signal processing;
a display unit configured to display the ultrasound image; and
a control panel with which an arbitrary parameter is set by an operator for generating the ultrasound image, wherein
a first reference position included in a first diagnosis region of the displayed ultrasound image is set by the control panel; and
the image generation unit is provided with a region generation portion for generating a second diagnosis region to be generated on the ultrasound image by using positional information of the first diagnosis region, protrusion to an outside of the ultrasound image, and edges and peripheral tissues of the first diagnosis region.
